(19) United States
(12) Patent Application Publication
(10) Pub. No.: US 2021/0220376 A1
(43) Pub. Date: Jul. 22, 2021

(54) ADDICTION TREATMENT OF AN ALCOHOL-CONSUMING PATIENT POPULATION

(71) Applicant: Amygdala Neurosciences, Inc., Palo Alto, CA (US)

(72) Inventors: Ivan Diamond, Berkeley, CA (US); Louis G. Lange, Palo Alto, CA (US); Peter M. Strumph, San Francisco, CA (US)

(73) Assignee: Amygdala Neurosciences, Inc., Palo Alto, CA (US)

(21) Appl. No.: 17/224,902

(22) Filed: Apr. 7, 2021

Related U.S. Application Data
(63) Continuation of application No. PCT/US19/55431, filed on Oct. 9, 2019.
(60) Provisional application No. 62/745,116, filed on Oct. 12, 2018.

(51) Int. Cl. A61K 31/675; A61K 31/4418; A61K 31/352; A61P 25/32

(52) U.S. Cl. CPC A61K 31/675; A61P 25/32; A61K 31/352; A61K 31/4418

(57) ABSTRACT

Disclosed herein are methods of treating addiction to a dopamine-producing agent (e.g., amphetamine, cocaine, nicotine, opioids) in patient populations that do not exclude alcohol consumption during treatment. The methods generally comprise administering to the patient a therapeutically effective amount of an aldehyde dehydrogenase-2 (ALDH-2) inhibitor, such as compound (1)

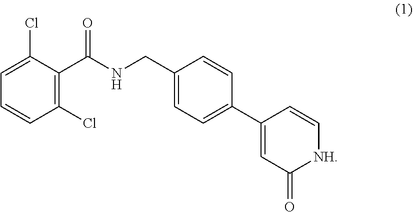 [SMILES: O=C(NCc1ccc(-c2cc[nH]c(=O)c2)cc1)c1c(Cl)cccc1Cl]

### CROSS-REFERENCE

This application is continuation of PCT/US19/55431, filed Oct. 9, 2019, which claims priority to U.S. Provisional Patent Application No. 62/745,116, filed Oct. 12, 2018, each of which is hereby incorporated by reference herein for all purposes.

### FIELD

The present disclosure relates to a method of treating addiction to a dopamine-producing agent (e.g., cocaine, nicotine, opioids) in a patient population that does not exclude alcohol consumption during treatment, the method comprising administering to the patient a therapeutically effective amount of an aldehyde dehydrogenase-2 (ALDH-2) inhibitor.

### BACKGROUND

Addiction remains a major health problem around the world. The United States Surgeon General has declared substance abuse a national health care crisis that is estimated to have resulted in greater than 3 months reduction in average U.S. life expectancy, 155,000 related deaths per year, 23 million needing treatment, and a $400 billion economic cost annually. See “Facing Addiction in America,” Surgeon General's Report, 2016. The Center for Disease Control estimates that illicit drug overdoses killed 64,000 people in the U.S. in 2016, with 14,000 of those deaths resulting from prescription opioid medications.

Inhibition of aldehyde dehydrogenase-2 (ALDH-2) has been shown to reduce pathophysiologic dopamine surge without changing basal dopamine levels in a rat model of cue-induced cocaine relapse-like behavior. See e.g., Yao et al., “Inhibition of aldehyde dehydrogenase-2 suppresses cocaine seeking by generating THP, a cocaine use-dependent inhibitor of dopamine synthesis,” Nature Medicine (2010), Vol. 16, No. 9; Diamond and Yao, “From Ancient Chinese Medicine to a Novel Approach to Treat Cocaine Addiction,” CNS & Neurological Disorders—Drug Targets (2015) Vol. 14, No. 6. A recent review concludes that dopamine surge above normal levels is part of the reward circuit common to all drugs of addiction. See e.g., Volkow et al., “Neurobiologic Advances from the Brain Disease Model of Addiction,” N. Engl. J. Med. (2016) 374:363-371.

The isoflavone compound, daidzein, and several of its structurally related derivatives have been shown to be selective inhibitors of ALDH-2, relative to the MAO pathway, and exhibit effectiveness in treating alcohol dependency. See e.g., Keung et al., (1993) Proc. Natl. Acad. Sci. USA 90, 10008-10012; Keung et al., (1997) Proc. Natl. Acad. Sci. USA 94, 1675-1679; U.S. Pat. Nos. 5,624,910, 6,121,010, 7,951,813, 8,158,810, and 8,673,966; International Patent Publ. Nos. WO2008/014497, WO2008/124532, WO2009/061924, WO2009/094028, and WO2013/033377.

A genus of compounds with a structural core unrelated to the isoflavones, such as 2,6-dichloro-N-[4-(2-oxo-1,2-dihydro-pyridin-4-yl)-benzyl]-benzamide (disclosed herein as compound (1)),

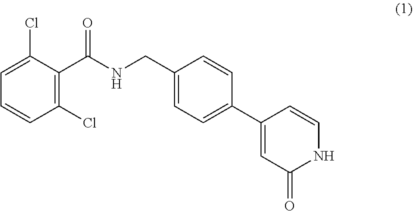 [SMILES: O=C(NCc1ccc(-c2cc[nH]c(=O)c2)cc1)c1c(Cl)cccc1Cl]

have been shown to inhibit ALDH-2 selectively relative to the monoamine oxidase (MAO) pathway, and exhibit effectiveness in treating rat models of alcohol, nicotine, and cocaine dependency. See e.g., U.S. Pat. Nos. 8,558,001, 8,575,353, 9,000,015, 9,610,299; Int'l Pat. Publ. WO2013/006400; and Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6.

Disulfuram (DSF) is an ALDH-2 inhibitor that has been approved by the FDA for the treatment of alcohol abuse. Alcohol consumption during treatment with DSF however results in potentially lethal cardiac side-effects including tachycardia, low blood pressure, and QTc prolongation, generally referred to as the disulfiram-ethanol reaction (DER). See e.g., Chick, “Safety issues concerning the use of disulfiram in treating alcohol dependence,” Drug Safety 20:427-435 (1999). The DER has been interpreted as resulting from inhibition of ALDH-2 in the liver of alcohol-consuming patients. See e.g., Chen et al., “Targeting Aldehyde Dehydrogenase 2: New Therapeutic Opportunities,” Physiol. Rev. 94: 1-34 (2014). Avoidance of the DER side-effects requires patients to abstain from alcohol consumption during DSF treatment, which was believed to create a further deterrent to alcohol consumption—a potential benefit in treating alcohol addiction. Id. However, attempts to treat addiction to cocaine with DSF have been stymied by the DER side-effects. For example, a safety study of DSF treatment for cocaine addiction in an alcohol-consuming patent population was halted prematurely due to severity of DER in patients receiving 500 mg/day DSF. Roache et al., “A Double-blind, Placebo-controlled Assessment of the Safety of Potential Interactions Between Intravenous Cocaine, Ethanol, and Oral Disulfiram,” Drug Alcohol Depend. 119 (1-2): 37-45 (2011).

In view of the well-known DER side-effects associated with addiction treatment of alcohol consuming patient populations with the ALDH-2 inhibitor DSF, there remains a need for improved therapeutic methods to treat addiction to dopamine-producing agents, such as nicotine, cocaine, and opioids, in patient populations that do not exclude intake of alcohol.

### SUMMARY

It is a surprising technical effect and unexpected advantage of the selective ALDH-2 inhibitors disclosed herein (e.g., compound (2)) can be used for the treatment of addiction in patients that consume alcohol without the dangerous cardiac side-effects associated with DSF treatment.

In some embodiments, the present disclosure provides methods of treating addiction to a dopamine-producing agent, the method comprising administering to a patient in need thereof, wherein the patient is a member of a patient population that does not exclude alcohol consumption during treatment, a therapeutically effective amount of an ALDH-2 inhibitor.

In some embodiments, the present disclosure provides an ALDH-2 inhibitor for use in treating addiction to a dopamine-producing agent in a patient, wherein the patient is a member of a patient population that does not exclude alcohol consumption during treatment.

In some embodiments, the present disclosure provides an ALDH-2 inhibitor for the manufacture of a medicament, wherein the medicament is for treating addiction to a dopamine-producing agent in a patient, wherein the patient is a member of a patient population that does not exclude alcohol consumption during treatment.

In some embodiments of the methods, uses, and manufactures disclosed herein, the patient consumes alcohol during treatment. In some embodiments, the patient consumes alcohol within about 1 hour, about 2 hours, about 3 hours, about 4 hours, or about 5 hours after administration of the ALDH-2 inhibitor.

In some embodiments of the methods, uses, and manufactures disclosed herein, the patient consumes alcohol in an amount of at least about 14 g, at least about 28 g, at least about 42 g, at least about 56 g, or at least about 70 g; optionally, the patient consumes alcohol in an amount of about 14 g to about 42 g, about 14 g to about 56 g, or about 14 g to about 70 g.

In some embodiments of the methods, uses, and manufactures disclosed herein, the therapeutically effective amount of the ALDH-2 inhibitor is at least 25 mg, at least 50 mg, at least 100 mg, at least 200 mg, at least 400 mg, or at least 600 mg; optionally, the therapeutically effective amount of the ALDH-2 inhibitor is about 25 mg to about 600 mg, about 50 mg to about 600 mg, about 25 mg to about 400 mg, about 25 mg to about 200 mg.

In some embodiments of the methods, uses, and manufactures disclosed herein, the ALDH-2 inhibitor is in a dosage form comprising the ALDH-2 inhibitor and a pharmaceutically acceptable carrier. In some embodiments of the methods, the ALDH-2 inhibitor is in an oral dosage form. In some embodiments of the methods, the ALDH-2 inhibitor is self-administered.

In some embodiments of the methods, uses, and manufactures disclosed herein, the dopamine-producing agent is an agent other than alcohol; optionally, the dopamine-producing agent is selected from amphetamine, cocaine, food, nicotine, opioids, or other drugs of addiction.

In some embodiments of the methods, uses, and manufactures disclosed herein, the patient population does not exclude male patients that consume from 1 to 5 alcoholic drinks during treatment. In some embodiments of the methods, the patient population does not exclude female patients that consume from 1 to 4 alcoholic drinks during treatment.

In the various embodiments of the methods, uses, and manufactures disclosed herein, the ALDH-2 inhibitor is a compound of Formula (I)

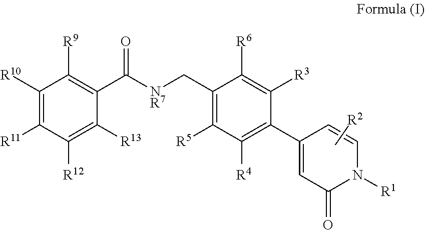

wherein:

R¹ is hydrogen, optionally substituted C₁₋₆ alkyl, —CH₂OH, —CH₂OP(O)(OR²⁰)(OR²¹);

R² is hydrogen, optionally substituted C₁₋₆ alkyl, cycloalkyl, or halo;

each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O—(C₁ to C₆-alkyl), cyano, halo, —SO₂NR²⁴R²⁵; or —NR²⁴R²⁵;

R⁷ is hydrogen or optionally substituted C₁₋₆ alkyl;

each of R²⁰ and R²¹ is independently Na⁺, Li⁺, K⁺, hydrogen, C₁₋₆ alkyl; or R²⁰ and R²¹ can be combined to represent a single divalent cation Zn²⁺, Ca²⁺, or Mg²⁺; and

each of R²⁴ and R²⁵ is independently chosen from hydrogen or C₁₋₆ alkyl or when combined together with the nitrogen to which they are attached form a heterocycle; or

a pharmaceutically acceptable salt, ester, single stereoisomer, mixture of stereoisomers, or a tautomer thereof.

In some embodiments of the methods, uses, and manufactures disclosed herein, the ALDH-2 inhibitor is a compound the compound of formula (I) is selected from the group consisting of: 2,6-dichloro-4-(2-methoxyethoxy)-N-(4-(2-oxo-1,2-dihydropyridin-4-yl) benzyl)benzamide; 2,6-dichloro-N-[4-(2-oxo-1,2-dihydro-pyridin-4-yl)-benzyl]-benzamide; 2-chloro-3-fluoro-N-(4-(2-oxo-1,2-dihydropyridin-4-yl)benzyl)benzamide; 2-chloro-6-methyl-N-(4-(2-oxo-1,2-dihydropyridin-4-yl)benzyl)benzamide; 2,6-dimethyl-N-(4-(2-oxo-1,2-dihydropyridin-4-yl)benzyl)benzamide; 2,6-dichloro-N-[4-(6-methyl-2-oxo-1,2-dihydro-pyridin-4-yl)-benzyl]-benzamide; 2-chloro-3,6-difluoro-N-(4-(2-oxo-1,2-dihydropyridin-4-yl)benzyl)benzamide; 2,6-dichloro-N-(3-methyl-4-(2-oxo-1,2-dihydropyridin-4-yl)benzyl)benzamide); 2,6-dichloro-N-(4-(1-methyl-2-oxo-1,2-dihydropyridin-4-yl)benzyl)benzamide; 2,6-difluoro-N-(4-(2-oxo-1,2-dihydropyridin-4-yl)benzyl)benzamide; 2-chloro-6-fluoro-N-(4-(2-oxo-1,2-dihydropyridin-4-yl)benzyl)benzamide; 2,6-dichloro-N-(2-fluoro-4-(2-oxo-1,2-dihydropyridin-4-yl)benzyl)benzamide; 2,6-dichloro-N-(4-(5-fluoro-2-oxo-1,2-dihydropyridin-4-yl)benzyl)benzamide; phosphoric acid mono-(4-{4-[(2,6-dichloro-benzoylamino)-methyl]-phenyl}-2-oxo-2H-pyridin-1-ylmethyl) ester; 2,6-dimethyl-N-(4-(2-oxopiperidin-4-yl)benzyl)benzamide; or a pharmaceutically acceptable salt, single stereoisomer, mixture of stereoisomers, or a tautomer thereof.

In some embodiments of the methods, uses, and manufactures disclosed herein, the ALDH-2 inhibitor is a compound of formula (I), wherein the compound of formula (I) is compound (1):

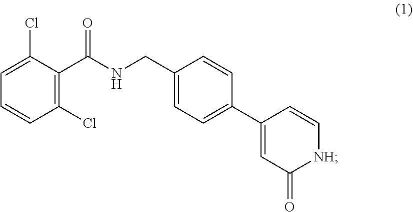 [SMILES: O=C(NCc1ccc(-c2cc[nH]c(=O)c2)cc1)c1c(Cl)cccc1Cl]

or a pharmaceutically acceptable salt, or a tautomer thereof.

In some embodiments of the methods and/or pharmaceutical compositions disclosed herein, the ALDH-2 inhibitor is a compound of formula (I), wherein the compound of formula (I) is compound (2):

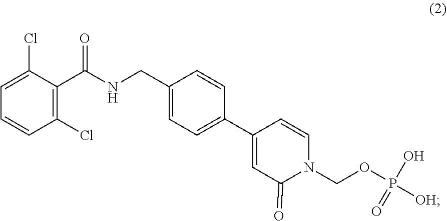 [SMILES: O=C(NCc1ccc(-c2ccn(COP(=O)(O)O)c(=O)c2)cc1)c1c(Cl)cccc1Cl]

or a pharmaceutically acceptable salt, ester, or a tautomer thereof.

In some embodiments of the methods, uses, and manufactures disclosed herein, the ALDH-2 inhibitor is a compound comprising an isoflavone structure. In some embodiments, the compound comprising an isoflavone structure is daidzein (compound (15)):

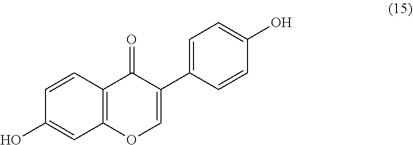 [SMILES: O=c1c(-c2ccc(O)cc2)coc2cc(O)ccc12]

or a pharmaceutically acceptable salt, ester, or a tautomer thereof. In some embodiments, the compound comprising an isoflavone structure is 3-{[3-(4-aminophenyl)-4-oxochromen-7-yloxy]methyl}benzoic acid (compound (16)):

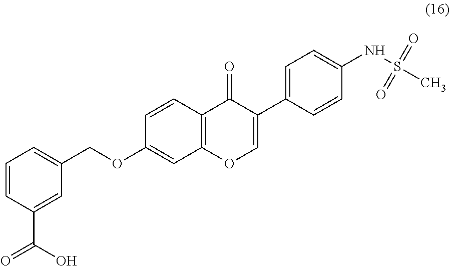 [SMILES: CS(=O)(=O)Nc1ccc(-c2coc3cc(OCc4cccc(C(=O)O)c4)ccc3c2=O)cc1]

or a pharmaceutically acceptable salt, ester, or a tautomer thereof.

Additional embodiments are described herein.

### DETAILED DESCRIPTION

It is to be understood that the detailed descriptions provided herein, including the drawings, are exemplary and explanatory only and are not restrictive of this disclosure. The description is not limited to the specific compounds, compositions, methods, techniques, protocols, cell lines, assays, and reagents disclosed herein, as these may vary, but is also intended to encompass known variants of these specific embodiments.

It is also to be understood that the terminology used herein is intended to describe particular embodiments and is in not intended to limit the scope as set forth in the appended claims. For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Further, it is understood that where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50” includes “2 to 25”, “5 to 20”, “25 to 50”, “1 to 10”, etc.

### Abbreviations, Definitions and General Parameters

As used in the present specification, the following words and phrases are generally intended to have the meanings as set forth below, except to the extent that the context in which they are used indicates otherwise.

The term “ALDH-2 inhibitor” as used herein includes any compound that selectively inhibits the enzyme aldehyde dehydrogenase 2. Exemplary ALDH-2 inhibitor compounds include the isoflavone compound, daidzein (see e.g., U.S. Pat. Nos. 5,624,910, and 6,121,010), and its structurally related isoflavone derivative compounds (see e.g., U.S. Pat. Nos. 7,951,813, 8,158,810, and 8,673,966; Int'l Pat. Publ. Nos. WO2008/014497, WO2008/124532, WO2009/061924, WO2009/094028, and WO2013/033377), and compounds of Formula (I), which are structurally unrelated to the isoflavones, such as 2,6-dichloro-N-[4-(2-oxo-1,2-dihydro-pyridin-4-yl)-benzyl]-benzamide (see e.g., U.S. Pat. Nos. 8,558,001, 8,575,353, 9,000,015, 9,610,299; Int'l Pat. Publ. WO2013/006400).

The term “addiction” as used herein includes any substance use disorder including, but not limited to, substance misuse, substance dependence, substance addiction, and/or conditioned response behavior in a mammal resulting from a dopamine producing agent.

The term “dopamine producing agents” as used herein includes compounds capable of inducing a surge in dopamine levels in a mammal, including, but not limited to, opioids, amphetamines, alcohol, other drugs of addiction, foods (e.g., sugary foods), and nicotine.

The term “alcohol” as used herein in the context of dopamine producing agents that may be consumed by humans refers to ethanol (“EtOH”).

The term “therapeutically effective amount” refers to an amount that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “unit dosage form” refers to physically discrete units suitable as unitary dosages for human subjects and other mammals, each unit containing a predetermined quantity of active ingredient that produces the desired therapeutic effect, in association with a suitable pharmaceutical excipient (e.g., a tablet, capsule, or ampoule).

The term “active ingredient” refers to a compound in a pharmaceutical composition that has a pharmacological effect when administered to an organism (e.g., a mammal) and is intended to encompass not only the compound but also the pharmaceutically acceptable salts, pharmaceutically acceptable esters, hydrates, polymorphs, and prodrugs of such compound.

The term “prodrug” refers to a compound that includes a chemical group which, in vivo, can be converted and/or split off from the remainder of the molecule to provide for the active drug, a pharmaceutically acceptable salt thereof, or a biologically active metabolite thereof.

The term “treatment” or “treating” means any administration of a compound of the disclosure to a mammal having a disease or disorder, or a mammal susceptible to a disease or disorder, for purposes including:
(i) preventing the disease, that is, causing the clinical symptoms of the disease not to develop;
(ii) inhibiting the disease, that is, arresting the development of clinical symptoms; and/or
(iii) relieving the disease, i.e. causing the regression of clinical symptoms.

The term “during treatment” as used herein refers to the time period after administration of a therapeutically effective amount of a compound to a subject for treatment of a disease or disorder until the time at which the amount of the compound in the subject has decreased to a level below what is therapeutically effective.

The term “alkyl” refers to a monoradical branched or unbranched saturated hydrocarbon chain having from 1 to 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

(i) an alkyl group as defined above, having 1, 2, 3, 4 or 5 substituents, (typically 1, 2, or 3 substituents) selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkyl group as defined above that is interrupted by 1-10 atoms (e.g. 1, 2, 3, 4, or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
(iii) an alkyl group as defined above that has both 1, 2, 3, 4 or 5 substituents as defined above and is also interrupted by 1-10 atoms (e.g. 1, 2, 3, 4, or 5 atoms) as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (typically 1, 2, or 3 substituents), as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, typically having from 1 to 20 carbon atoms (e.g. 1-10 carbon atoms, or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, typically having 1, 2, 3, 4, 5, or 6 carbon atoms.

The term “substituted alkylene” refers to:
(i) an alkylene group as defined above having 1, 2, 3, 4, or 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(ii) an alkylene group as defined above that is interrupted by 1-10 groups (e.g. 1, 2, 3, 4, or 5 groups) independently chosen from —O—, —S—, sulfonyl, —C(O)—, —C(O)O—, —C(O)N—, and —NR^{a}, where R^{a} is chosen from hydrogen, optionally substituted alkyl, cycloalkyl, cycloalkenyl, aryl, heteroaryl and heterocyclyl; or
(iii) an alkylene group as defined above that has both 1, 2, 3, 4 or 5 substituents as defined above and is also interrupted by 1-10 groups as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂—N(CH₃)—CH₂CH₂—), 1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkoxy” refers to the group R—O—, where R is optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are alkyl-O— and include, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl as defined above. This term is exemplified by groups such as methoxy, ethoxy, n-propoxy, iso-propoxy, n-butoxy, iso-butoxy, t-butoxy, n-hexyloxy, and the like.

The term “alkylthio” refers to the group R—S—, where R is as defined for alkoxy.

The term “alkenyl” refers to a monoradical of a branched or unbranched unsaturated hydrocarbon group typically having from 2 to 20 carbon atoms (more typically from 2 to 10 carbon atoms, e.g. 2 to 6 carbon atoms) and having from 1 to 6 carbon-carbon double bonds, e.g. 1, 2, or 3 carbon-carbon double bonds. Typical alkenyl groups include ethenyl (or vinyl, i.e. —CH═CH₂), 1-propylene (or allyl, —CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, typically having from 2 to 20 carbon atoms (more typically from 2 to 10 carbon atoms, e.g. 2 to 6 carbon atoms) and having from 1 to 6 carbon-carbon triple bonds e.g. 1, 2, or 3 carbon-carbon triple bonds. Typical alkynyl groups include ethynyl (—C≡CH), propargyl (or propynyl, —C≡CH₃), and the like. In the event alkynyl is attached to nitrogen, the triple bond cannot be alpha to the nitrogen.

The term “substituted alkynyl” refers to an alkynyl group as defined above having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, cycloalkyl, aryl, heteroaryl, heterocyclyl or where both R groups are joined to form a heterocyclic group (e.g., morpholino). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “ester” or “carboxyester” refers to the group —C(O)OR, where R is alkyl, cycloalkyl, aryl, heteroaryl, or heterocyclyl, which may be optionally further substituted by alkyl, alkoxy, halogen, CF₃, amino, substituted amino, cyano, or —S(O)ₙR^{a}, in which R^{a} is alkyl, aryl, or heteroaryl and n is 0, 1 or 2.

The term “acylamino” refers to the group —NRC(O)R where each R is independently hydrogen, alkyl, aryl, heteroaryl, or heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2.

The term “acyloxy” refers to the groups —OC(O)-alkyl, —OC(O)-cycloalkyl, —OC(O)-aryl, —OC(O)-heteroaryl, and —OC(O)-heterocyclyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl, fluorenyl, and anthryl). Typical aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl, —C(O)O-cycloalkyl, where alkyl and cycloalkyl are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and bicyclo[2.2.1]heptane, or cyclic alkyl groups to which is fused an aryl group, for example indan, and the like.

The term “cycloalkenyl” refers to cyclic alkyl groups of from 3 to 20 carbon atoms having a single cyclic ring or multiple condensed rings and having at least one double bond and preferably from 1 to 2 double bonds.

The terms “substituted cycloalkyl” and “substituted cycloalkenyl” refer to cycloalkyl or cycloalkenyl groups having 1, 2, 3, 4 or 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl. The term “substituted cycloalkyl” also includes cycloalkyl groups wherein one or more of the annular carbon atoms of the cycloalkyl group is a carbonyl group (i.e. an oxygen atom is oxo to the ring). Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “alkoxycarbonylamino” refers to a group —NHC(O)OR in which R is optionally substituted alkyl.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which R each R is independently an optionally substituted alkyl.

The term “azido” refers to a group

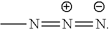

The term “hydroxyl” or “hydroxyl” refers to a group —OH.

The term “arylthio” refers to the group —S-aryl.

The term “heterocyclylthio” refers to the group —S-heterocyclyl.

The term “alkylthio” refers to the group —S-alkyl.

The term “aminosulfonyl” refers to the group —SO₂NRR, wherein each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclyloxy, hydroxyamino, alkoxyamino, nitro, —SO-alkyl, —SO-aryl, —SO-heteroaryl, —SO₂-alkyl, SO₂-aryl and —SO₂-heteroaryl.

The term “aminocarbonylamino” refers to the group —NR^{c}(O)NRR, wherein R^{c} is hydrogen or alkyl and each R is independently selected from the group consisting of hydrogen, alkyl, cycloalkyl, aryl, heteroaryl and heterocyclyl. Unless otherwise constrained by the definition, all substituents may optionally be further substituted by 1, 2, or 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl.

The term “heterocyclooxy” refers to the group —O-heterocyclyl.

The term “alkoxyamino” refers to the group —NHOR in which R is optionally substituted alkyl.

The term “hydroxyamino” refers to the group —NHOH.

The term “heteroaryl” refers to a group comprising single or multiple rings comprising 1 to 15 carbon atoms and 1 to 4 heteroatoms selected from oxygen, nitrogen, and sulfur within at least one ring. The term “heteroaryl” is generic to the terms “aromatic heteroaryl” and “partially saturated heteroaryl.” The term “aromatic heteroaryl” refers to a heteroaryl in which at least one ring is aromatic. Examples of aromatic heteroaryls include pyrrole, thiophene, pyridine, quinoline, pteridine. The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, and the like.

Unless otherwise constrained by the definition for the heteroaryl substituent, such heteroaryl groups can be optionally substituted with 1 to 5 substituents (typically 1, 2, or 3 substituents) selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl (an alkyl ester), arylthio, heteroaryl, heteroarylthio, heterocyclylthio, thiol, alkylthio, aryl, aryloxy, aralkyl, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Such heteroaryl groups can have a single ring (e.g., pyridyl or furyl) or multiple condensed rings (e.g., indolizinyl, benzothiazole, or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “heterocyclyl,” “heterocycle,” or “heterocyclic” refers to a monoradical saturated group having a single ring or multiple condensed rings, having from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5 substituents (typically 1, 2, or 3 substituents), selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2. Preferred heterocyclics include tetrahydrofuranyl, morpholino, piperidinyl, and the like.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” or “oxo” refers to a group —C(O)—.

The term “thiocarbonyl” refers to a group —C(S)—.

The term “carboxy” refers to a group —C(O)—OH.

The term “optional” or “optionally” mean that the subsequently described event or circumstance may or may not occur, and that the description includes instances where said event or circumstance occurs and instances in which it does not.

The term “substituted” includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to substituted aryl-(substituted aryl)-substituted aryl.

A compound of a given formula (e.g. the “compound of Formula (I)”) is intended to encompass the compounds of the disclosure, and the pharmaceutically acceptable salts, pharmaceutically acceptable esters, hydrates, polymorphs, and prodrugs of such compounds.

Additionally, the compounds of the disclosure may possess one or more asymmetric centers and can be produced as a racemic mixture or as individual enantiomers or diastereoisomers. The number of stereoisomers present in any given compound of a given Formula depends upon the number of asymmetric centers present (there are 2n stereoisomers possible where n is the number of asymmetric centers). The individual stereoisomers may be obtained by resolving a racemic or non-racemic mixture of an intermediate at some appropriate stage of the synthesis, or by resolution of the compound by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

The term “isomers” means different compounds that have the same molecular formula. Isomers include stereoisomers, enantiomers, and diastereomers.

The term “stereoisomers” means isomers that differ only in the way the atoms are arranged in space.

The term “enantiomers” means a pair of stereoisomers that are non-superimposable mirror images of each other. A 1:1 mixture of a pair of enantiomers is a “racemic” mixture. The term “(±)” is used to designate a racemic mixture where appropriate.

The term “diastereoisomers” means stereoisomers that have at least two asymmetric atoms, but which are not mirror-images of each other.

Absolute stereochemistry is specified herein according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or levorotary) that they rotate the plane of polarized light at the wavelength of the sodium D line.

Some of the compounds of the present disclosure exist as “tautomeric isomers” or “tautomers.” “Tautomeric isomers” or “tautomers” are isomers that are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. Non-limiting examples of amide-comprising and imidic acid-comprising tautomers are shown below:

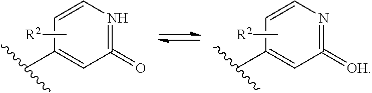

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by combining a compound and a solvent.

The term “hydrate” refers to the complex formed by combining a compound and water.

The term “pharmaceutically acceptable salt” of a given compound refers to salts that retain the biological effectiveness and properties of the given compound, and which are not biologically or otherwise undesirable. In many cases, the compounds of this disclosure are capable of forming pharmaceutically acceptable acid and/or base salts by virtue of the presence of amino and/or carboxyl groups or groups similar thereto.

Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts also may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terms “pharmaceutically acceptable carrier” or “pharmaceutically acceptable excipient” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Any formula or structure given herein, including Formula (I) compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium) ¹¹C, ¹³C, ¹⁴C, ¹⁵N, ¹⁸F, ³¹F, ³²F, ³⁵P, ³⁶Cl, and ¹²⁵I. Various isotopically labeled compounds of the present invention, for example those into which radioactive isotopes such as ³H, ¹³C, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients.

Deuterium labelled or substituted therapeutic compounds of the invention may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula (I).

The concentration of such a heavier isotope, specifically deuterium, may be defined by an isotopic enrichment factor. In the compounds of this invention any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this invention any atom specifically designated as a deuterium (D) is meant to represent deuterium.

In the description, including the examples, all temperatures are in degrees Celsius (° C.), unless otherwise stated, and abbreviations and acronyms have the following meanings:

| Abbreviation | Meaning |
|---|---|
| ° C. | Degree Celsius |
| 5-HIAA | 5-Hydroxyindoleacetic acid |
| 5-HIAL | 5-Hydroxyindoleacetaldehyde |
| 5-HT | 5-Hydroxytryptamine (serotonin) |
| 5-HTOL | 5-Hydroxytryptophol |
| Ae | Enzyme activities measured in the presence of a test |
| | compound |
| AIDS | Acquired immune deficiency syndrome |
| ALDH-2 | Human mitochondrial aldehyde dehydrogenase |
| Ao | Enzyme activities measured in the absence of a test |
| | compound |
| BHA | Butylated hydroxy anisole |
| BOC | tert-Butoxycarbonyl |
| BOP | Benzotriazolyl-N-hydroxytris(dimethyamino)phosphonium |
| | hexafluorophosphate |
| Cbz | Benzyl carbamate |
| cm | centimeter |
| d | Doublet |
| dd | Doublet of doublets |
| DA | Dopamine |
| DCC | Dicyclohexyl carbodiimide |
| DCM | Dichloromethane |
| DIC | Diisopropyl carbodiimide |
| DIEA | N,N-Diisopropylethylamine |
| DMF | Dimethylformamide |
| DMSO | Dimethylsulfoxide |
| dt | Doublet of triplets |
| EDTA | Ethylenediaminetetraacetic acid |
| equiv/eq | Equivalents |
| EtOAc | Ethyl acetate |
| EtOH | Ethanol |
| FR | Fixed ratio |
| g | Grams |
| HATU | O-(7-Azabenzotriazol-1-yl)-N,N,N′,N′-tetramethyluronium |
| | hexafluorophosphate |
| HBTU | O-Benzotriazole-N,N,N′,N′-tetramethyl-uronium- |
| | hexafluoro-phosphate |
| HPLC | High-performance liquid chromatography |
| hrs/h | Hours |
| Hz | Hertz |
| IC50 | The half maximal inhibitory concentration |
| IIDQ | 1-Isobutoxycarbonyl-2-isobutoxy-1,2-dihydro quinone |
| ip | Intraperitoneal |
| iv | Intravenous |
| J | Coupling constant |
| Kg | Kilogram |
| L | Liter |
| LAD | Low alcohol-drinking rat |
| LCMS/LC-MS | Liquid chromatography-mass spectrometry |
| LG | Leaving group |
| M | Molar |
| m/z | mass-to-charge ratio |
| M+ | Mass peak |
| M + H | Mass peak plus hydrogen |
| M + Na | Mass peak plus sodium |
| MAO | Monoamine oxidase |
| Me | Methyl |
| mg | Milligram |
| MHz | Megahertz |
| min | Minute |
| ml/mL | Milliliter |
| mM | Millimolar |
| mmol | Millimole |
| MOM | Methoxylmethyl |
| MS | Mass spectroscopy |
| NAD | Nicotinamide Adenine Dinucleotide |
| NaPPi | Sodium pyrophosphate |
| NIH | National Institute of Health |
| NMM | N-Methylmorpholine |
| NMR | Nuclear magnetic resonance |
| NP | Alcohol non-preferring rat |
| OCD | Obsessive compulsive disorder |
| PG | Protecting group |
| Ph | Phenyl |
| PyBOP | (Benzotriazol-1-yloxy)tripyrrolidinophosphonium |
| | hexafluorophosphate |
| q.s. | Quantity sufficient to achieve a stated function |
| RT/rt/R.T | Room temperature |
| s | Second |
| s | Singlet |
| SA | Self-administration |
| sc | Subcutaneous |
| SEM | Standard error of means |
| t | Triplet |
| TEA | Triethylamine |
| TES | Triethylsilyl |
| TFA | Trifluoroacetic acid |
| THF | Tetrahydrofuran |
| TIPS | Triisopropylsilyl |
| TKK | TKK buffer |
| TLC | Thin layer chromatography |
| TMS | Trimethylsilyl |
| TO | Time out |
| Tris | tris(hydroxymethyl)aminomethane |
| δ | Chemical shift |
| μg | Microgram |
| μL/μl | Microliter |
| μM | Micromolar |
| μmol | Micromole |

Dopamine-Producing Agents and Addiction

Dopamine-producing agents that are well-known for their addictive characteristics include alcohol, amphetamines, cocaine, nicotine, opioids, other drugs of addiction, and foods (e.g., sugary foods). It is well-established that these dopamine-producing agents when administered to mammals (e.g., humans) induce surges in dopamine levels (either directly or indirectly) that can result in the acquisition of a conditioned response leading to the deleterious side-effect of addiction (e.g., misuse, dependence, abuse). For example, nicotine exerts its dopamine-producing effect by binding to neuronal nicotinic acetylcholine receptors (nAChRs). Presynaptic nAChRs on midbrain dopamine neurons project from the ventral tegmental area to the nucleus accumbens and prefrontal cortex. These presynaptic nAChRs induce dopamine release when activated by nicotine. Sacco et al., “Nicotinic receptor mechanisms and cognition in normal states and neuropsychiatric disorders,” J. Psychopharmacol. 18:457-474 (2004).

It is well-known that abuse of many dopamine producing agents, for example nicotine, and cocaine, commonly occurs concurrent with the use (and abuse) of alcohol. It also has been found that the particular combination of cocaine and alcohol exerts more cardiovascular toxicity in humans than either drug alone. However, in treating addiction to dopamine producing agents, particularly nicotine and/or cocaine, it is often very difficult for the patient to fully abstain from alcohol. Indeed, the inability to adhere to alcohol exclusion during treatment can lead to withdrawal from treatment altogether and subsequent relapse.

ALDH-2 Inhibitor Compounds

Compounds that act as selective inhibitors of ALDH-2 have been shown to reduce pathophysiologic dopamine surge without changing basal dopamine levels. See e.g., Yao et al., “Inhibition of aldehyde dehydrogenase-2 suppresses cocaine seeking by generating THP, a cocaine use-dependent inhibitor of dopamine synthesis,” Nature Medicine (2010), Vol. 16, No. 9; Diamond and Yao, “From Ancient Chinese Medicine to a Novel Approach to Treat Cocaine Addiction,” CNS & Neurological Disorders—Drug Targets (2015) Vol. 14, No. 6. Selective inhibitors of ALDH-2 have also demonstrated the ability to suppress self-administration of nicotine in rats. See e.g., Rezvani et al., “Inhibition of Aldehyde Dehydrogenase-2 (ALDH-2) Suppresses Nicotine Self-Administration in Rats,” (2015) Journal of Drug and Alcohol Research, vol. 4: 1-6; and U.S. Pat. Nos. 8,558,001, 8,575,353, 9,000,015, 9,610,299; Int'l Pat. Publ. WO2013/006400.

The ALDH-2 inhibitor compounds provided in the present disclosure have been shown to be useful in methods for the reduction and/or prevention of addiction in mammals to dopamine-producing agents including alcohol, cocaine, and nicotine. ALDH-2 inhibitor compounds useful in the methods, uses and manufactures of the present disclosure can include any of the compounds well-known in the art as ALDH-2 inhibitors including, but not limited to, daidzein (compound (15)), or its pharmaceutically acceptable salts, esters, or a tautomer thereof.

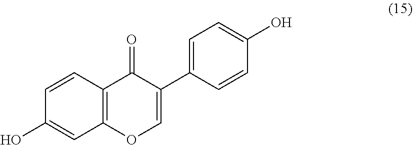 [SMILES: O=c1c(-c2ccc(O)cc2)coc2cc(O)ccc12]

ALDH-2 inhibitor compounds useful in the methods, uses and manufactures of the present disclosure can include the isoflavone compounds structurally related to daidzein, such as 3-{[3-(4-aminophenyl)-4-oxochromen-7-yloxy]methyl}benzoic acid (compound (16)), or its pharmaceutically acceptable salts, esters, or a tautomer thereof.

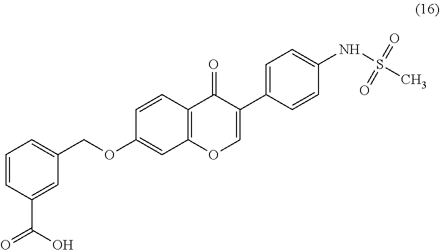 [SMILES: CS(=O)(=O)Nc1ccc(-c2coc3cc(OCc4cccc(C(=O)O)c4)ccc3c2=O)cc1]

Additional ALDH-2 inhibitor compounds comprising an isoflavone structure that are useful in the methods and compositions of the present disclosure are described in U.S. Pat. Nos. 5,624,910, 6,121,010 7,951,813, 8,158,810, and 8,673,966, and Int'l Pat. Publ. Nos. WO2008/014497, WO2008/124532, WO2009/061924, WO2009/094028, and WO2013/033377, each of which is hereby incorporated by reference herein.

ALDH-2 inhibitor compounds useful in the methods, uses and manufactures of the present disclosure can include any of the ALDH-2 inhibitor compounds that are structurally unrelated to daidzein and the other isoflavones. These include the ALDH-2 inhibitor compounds described in U.S. Pat. Nos. 8,558,001, 8,575,353, 9,000,015, 9,610,299, Int'l Pat. Publ. WO2013/006400, each of which is hereby incorporated by reference herein. Accordingly, in some embodiments of the methods, uses and manufactures of the present disclosure, the ALDH-2 inhibitor is a compound of Formula (I):

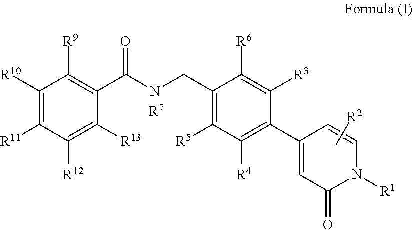

wherein:

R¹ is hydrogen, optionally substituted C₁₋₆ alkyl, —CH₂OH, —CH₂OP(O)(OR²⁰)(OR²¹);

R² is hydrogen, optionally substituted C₁₋₆ alkyl, cycloalkyl, or halo;

each of R³, R⁴, R⁵, R⁶, R⁹, R¹⁶, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O—(C₁ to C₆-alkyl), cyano, halo, —SO₂NR²⁴R²⁵; or —NR²⁴R²⁵;

R⁷ is hydrogen or optionally substituted C₁₋₆ alkyl;

each of R²⁰ and R²¹ is independently Na⁺, K⁺, hydrogen, C₁₋₆ alkyl; or R²⁰ and R²¹ can be combined to represent a single divalent cation Zn²⁺, Ca²⁺, or Mg²⁺; and

each of R²⁴ and R²⁵ is independently chosen from hydrogen or C₁₋₆ alkyl or when combined together with the nitrogen to which they are attached form a heterocycle; or

a pharmaceutically acceptable salt, ester, single stereoisomer, mixture of stereoisomers, or a tautomer thereof.

The naming and numbering of the compounds of Formula (I) is illustrated with a representative compound (1):

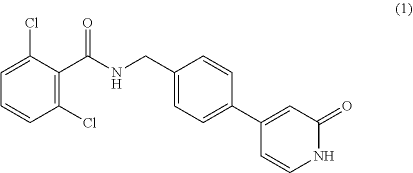 [SMILES: O=C(NCc1ccc(-c2cc[nH]c(=O)c2)cc1)c1c(Cl)cccc1Cl]

namely: 2,6-dichloro-N-[4-(2-oxo-1,2-dihydro-pyridin-4-yl)-benzyl]-benzamide.
In certain embodiments, R¹ is hydrogen. In certain embodiments, R¹ is C₁₋₆ alkyl. In certain embodiments, R¹ is methyl. In certain embodiments, R¹ is —CH₂OP(O)(OR20)(OR21); and each of R²⁰ and R²¹ is independently Na⁺, Li⁺, K⁺, or hydrogen. In certain embodiments, at least one of R¹, R⁹, R¹⁰, R¹¹, R¹², R¹³, is not hydrogen. In other embodiments, at least two of R¹, R⁹, R¹⁰, R¹¹, R¹², R¹³ is not hydrogen.

In certain embodiments, R² is hydrogen. In certain embodiments, R² is C₁₋₆ alkyl. In certain embodiments, R² is methyl. In certain embodiments, R² is selected from the group consisting of ethyl, n-propyl, iso-propyl, n-butyl, iso-butyl, t-butyl, and n-hexyl. In certain embodiments, R² is halo. In certain embodiments, R² is fluoro. In certain embodiments, R² is chloro. In certain embodiments, R² is bromo. In certain embodiments, R² is iodo.

In certain embodiments, each of R³, R⁴, R⁵, R⁶, R⁹, R¹⁰, R¹¹, R¹² and R¹³, is independently hydrogen, hydroxyl, —OP(O)(OR²⁰)(OR²¹), —CH₂OH, —CH₂OP(O)(OR²⁰)(OR²¹), optionally substituted C₁₋₆ alkyl, optionally substituted C₃₋₈ cycloalkyl, optionally substituted C₁₋₆ alkoxy, —O—(C₁ to C₆-alkyl)-O—(C₁ to C₆-alkyl), —C(O)NH₂, cyano, or halo. In certain embodiments, each of R³, R⁴, R⁵, and R⁶ is independently hydrogen, C₁₋₆ alkyl, or halo. In certain embodiments, one of R³, R⁴, R⁵, or R⁶ is C₁₋₆ alkyl or halo. In certain embodiments, one of R³, R⁴, R⁵, or R⁶ is selected from the group consisting of ethyl, n-propyl, iso-propyl, n-butyl, iso-butyl, t-butyl, and n-hexyl. In certain embodiments, one of R³, R⁴, R⁵, or R⁶ is methyl. In certain embodiments, one of R³, R⁴, R⁵, or R⁶ is fluoro. In certain embodiments, one of R³, R⁴, R⁵, or R⁶ is chloro. In certain embodiments, one of R³, R⁴, R⁵, or R⁶ is fluoro. In certain embodiments, one of R³, R⁴, R⁵, or R⁶ is iodo.

In certain embodiments, R⁷ is hydrogen. In certain embodiments, R⁷ is C₁₋₆ alkyl. In certain embodiments, R⁷ is selected from the group consisting of ethyl, n-propyl, iso-propyl, n-butyl, iso-butyl, t-butyl, and n-hexyl. In certain embodiments, R⁷ is methyl.

In certain embodiments, at least one of R⁹ and R¹³ is not hydrogen. In certain embodiments, at least one of R⁹ and R¹³ is halo or C₁₋₆ alkyl. In certain embodiments, at least one of R⁹ and R¹³ is selected from the group consisting of ethyl, n-propyl, iso-propyl, n-butyl, iso-butyl, t-butyl, and n-hexyl. In certain embodiments, at least one of R⁹ and R¹³ is independently chloro, fluoro, or methyl. In certain embodiments, at least one of R⁹ and R¹³ is bromo. In certain embodiments, at least one of R⁹ and R¹³ is iodo. In certain embodiments, R⁹ and R¹³ are independently halo or C₁₋₆ alkyl. In certain embodiments, R⁹ and R¹³ are independently chloro, fluoro, or methyl. In certain embodiments, R⁹ and R¹³ are chloro. In certain embodiments, R⁹ and R¹³ are methyl.

In certain embodiments, each of R¹⁰ and R¹² is independently hydrogen, halo, or C₁₋₆ alkyl. In certain embodiments, each of R¹⁰ and R¹² is independently ethyl, n-propyl, iso-propyl, n-butyl, iso-butyl, t-butyl, and n-hexyl. In certain embodiments, each of R¹⁰ and R¹² is independently hydrogen, chloro, fluoro, or methyl. In certain embodiments, each of R¹⁰ and R¹² is independently bromo. In certain embodiments, each of R¹⁰ and R¹² is independently iodo. In certain embodiments, each of R¹⁰ and R¹² is independently fluoro. In certain embodiments, each of R¹⁰ and R¹² is independently chloro. In certain embodiments, R¹⁰ and R¹² are hydrogen.

In certain embodiments, R¹¹ is hydrogen. In certain embodiments, R¹¹ is —O—(C₁ to C₆-alkyl)-O—(C₁ to C₆-alkyl). In certain embodiments, R¹¹ is —OCH₂CH₂OCH₃. In certain embodiments, R¹¹ is independently ethyl, n-propyl, iso-propyl, n-butyl, iso-butyl, t-butyl, and n-hexyl. In certain embodiments, R¹¹ is halo. In certain embodiments, R¹¹ is fluoro. In certain embodiments, R¹¹ is chloro. In certain embodiments, R¹¹ is bromo. In certain embodiments, R¹¹ is iodo.

In certain embodiments,

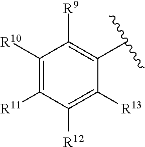

is selected from the group consisting of:

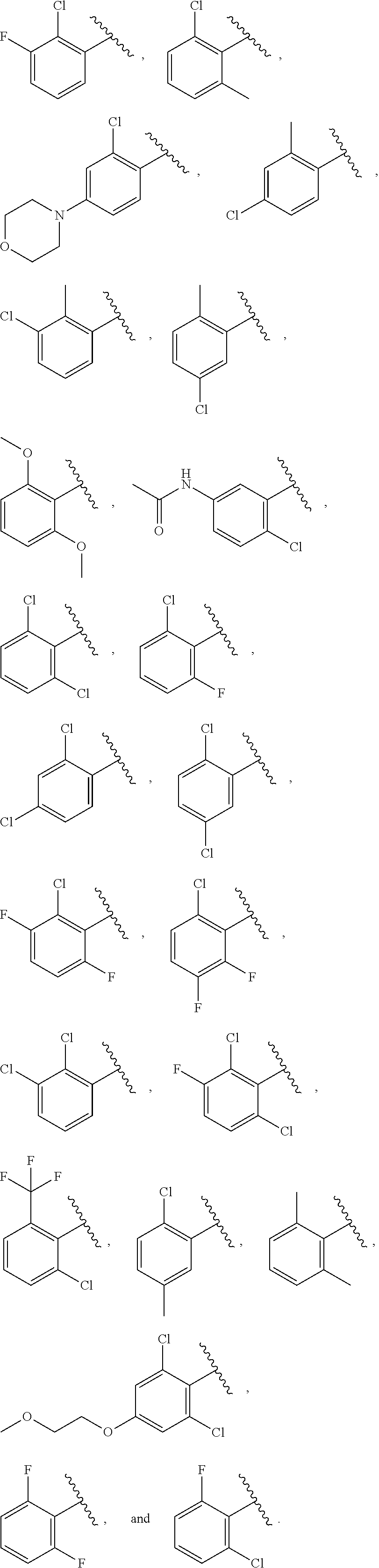

In certain embodiments, R¹ is hydrogen, methyl, or —CH₂OP(O)(OR²⁰)(OR²¹); R² is hydrogen, methyl, or fluoro; each of R³ and R⁴ is independently hydrogen or methyl; each of R⁵ and R⁶ is independently hydrogen or fluoro; R⁷ is hydrogen; R⁹ is hydrogen, chloro, fluoro, or methyl; R¹⁰ is hydrogen or fluoro; R¹¹ is hydrogen or —OCH₂CH₂OCH₃; R¹² is hydrogen or fluoro; R¹³ is hydrogen, chloro, fluoro, or methyl; and each of R²⁰ and R²¹ is independently Na⁺, Li⁺, K⁺, or hydrogen.

In certain embodiments, the ALDH-2 inhibitor compound of Formula (I) is selected from the group consisting of the compounds (1)-(14) listed in Table 1 (below). As described in U.S. Pat. No. 8,558,001, each of these compounds exhibits high, selective inhibition of the human ALDH-2 enzyme, with IC₅₀ values of less than 1 μm, and relatively low inhibitory activity toward the MAO-A and MAO-B pathway enzymes, with IC₅₀ values of >130 μm. It should be noted that high IC₅₀ value for compound (2) is due to it being a phosphoric acid adduct prodrug of compound (1). Thus, compound (2) undergoes in vivo cleavage of the phosphoric acid group to yield compound (1).

TABLE 1
Exemplary ALDH-2 Inhibitor Compounds of Formula (I)

| | | IC50 | IC50 | IC50 |
|---|---|---|---|---|
| Compound | | ALDH-2 | hMAO-A | hMAO-B |
| No. | Compound Name | (nm) | (μm) | (μm) |
| (1) | 2,6-dichloro-N-[4-(2-oxo-1,2-dihydro- | 102 | >130 | >130 |
| | pyridin-4-yl)-benzyl]-benzamide | | | |
| (2) | phosphoric acid mono-(4-{4-[(2,6-dichloro- | >10000.00 | >129.51 | >130 |
| | benzoylamino)-methyl]-phenyl]-2-oxo-2H- | | | |
| | pyridin-1-ylmethyl) ester | | | |
| (3) | 2,6-dichloro-4-(2-methoxyethoxy)-N-(4-(2- | 63 | >130 | >130 |
| | oxo-1,2-dihydropyridin-4-yl)benzyl)benzamide | | | |
| (4) | 2-chloro-3-fluoro-N-(4-(2-oxo-1,2-dihydropyridin- | 215 | >130 | >130 |
| | 4-yl)benzyl)benzamide | | | |
| (5) | 2-chloro-6-methyl-N-(4-(2-oxo-1,2- | 23 | >130 | >130 |
| | dihydropyridin-4-yl)benzyl)benzamide | | | |
| (6) | 2,6-dimethyl-N-(4-(2-oxo-1,2-dihydropyridin- | 166 | >130 | >130 |
| | 4-yl)benzyl)benzamide | | | |
| (7) | 2,6-dichloro-N-[4-(6-methyl-2-oxo-1,2-dihydro- | 1113 | >130 | >130 |
| | pyridin-4-yl)-benzyl]-benzamide | | | |
| (8) | 2-chloro-3,6-difluoro-N-(4-(2-oxo-1,2-dihydropyridin- | 464 | >130 | >130 |
| | 4-yl)benzyl)benzamide | | | |
| (9) | 2,6-dichloro-N-(3-methyl-4-(2-oxo-1,2-dihydropyridin- | 480 | >130 | >130 |
| | 4-yl)benzyl)benzamide | | | |
| (10) | 2,6-dichloro-N-(4-(1-methyl-2-oxo-1,2-dihydropyridin- | 2093 | >130 | >130 |
| | 4-yl)benzyl)benzamide | | | |
| (11) | 2,6-difluoro-N-(4-(2-oxo-1,2-dihydropyridin- | 890 | >130 | >130 |
| | 4-yl)benzyl)benzamide | | | |
| (12) | 2-chloro-6-fluoro-N-(4-(2-oxo-1,2-dihydropyridin- | 379 | >130 | >130 |
| | 4-yl)benzyl)benzamide | | | |
| (13) | 2,6-dichloro-N-(2-fluoro-4-(2-oxo-1,2-dihydropyridin- | 304 | >130 | >130 |
| | 4-yl)benzyl)benzamide | | | |
| (14) | 2,6-dichloro-N-(4-(5-fluoro-2-oxo-1,2-dihydropyridin- | 25 | >130 | >130 |
| | 4-yl)benzyl)benzamide | | | |

In certain embodiments, the compound of Formula (I) is compound (1):

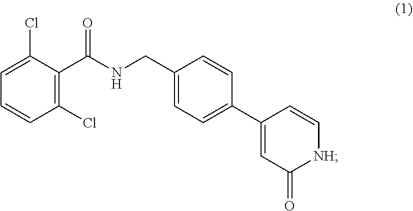 [SMILES: O=C(NCc1ccc(-c2cc[nH]c(=O)c2)cc1)c1c(Cl)cccc1Cl]

or a pharmaceutically acceptable salt, ester, single stereoisomer, mixture of stereoisomers, or tautomer thereof.

In certain embodiments, the compound of Formula (I) is compound (2):

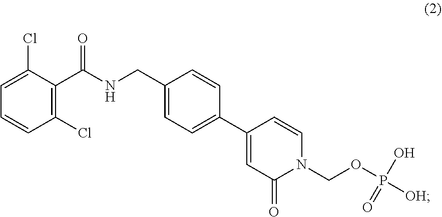 [SMILES: O=C(NCc1ccc(-c2ccn(COP(=O)(O)O)c(=O)c2)cc1)c1c(Cl)cccc1Cl]

or a pharmaceutically acceptable salt, ester, single stereoisomer, mixture of stereoisomers, or tautomer thereof. As noted above, compound (2) is an exemplary prodrug compound of Formula (I). In vivo, compound (2) generates the free amide (pyridine) compound (1) as a metabolite. Accordingly, one of ordinary skill in the art can synthesize other prodrugs of compounds of Formula (I) based on the disclosure herein and synthetic methods well-known in the art.

Preparation of ALDH-2 Inhibitor Compounds of Formula (I)

The ALDH-2 inhibitor compounds of Formula (I) can be prepared from readily available starting materials using methods and procedures known in the art. In particular, the disclosure of U.S. Pat. No. 8,558,001 (Cannizzaro et al.) issued Oct. 15, 2013, which is hereby incorporated by reference herein, provides general synthetic strategies for preparing compounds of Formula (I), and also exemplifies specific synthesis protocols that can be used to prepare the compounds (1), (2), (3), (4), (5), (6), (7), (8), (9), (10), (11), (12), (13), and (14) described herein and listed above in Table 1. Further, the synthetic protocol for the preparation of compounds (1) and (2) is provided below in the Examples of the present disclosure.

Briefly, the compounds of Formula (I) may be prepared according to the synthetic sequence shown in Scheme I

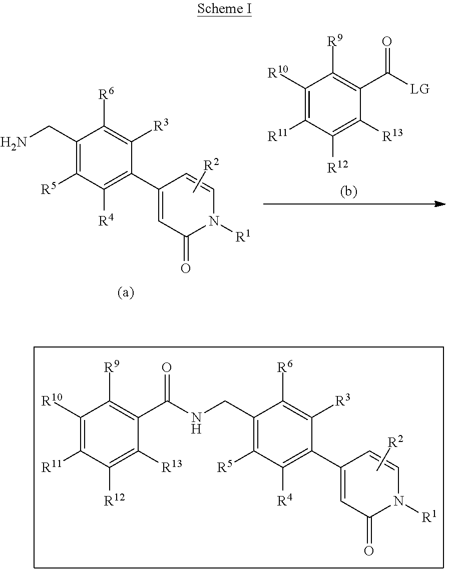

wherein, substituents R¹ through R²⁷, X¹, Y¹, Z² and are as defined herein; LG is a leaving group (e.g., Z² halo, hydroxyl, alkoxy, OSO₂ CF₃, N₂⁺, etc.); PG is a protecting group (e.g., t-butyl, t-butyl carbamate (BOC), etc.); and Z² is (OH)₂, (OMe)₂, F³⁻, or (OR^{H})(OR^{J}), wherein OR^{H} and OR^{J} may combine with boron to form a cyclic arylboronic ester moiety or cyclic alkylboronic ester moiety as described herein (e.g., 4,4,5,5-tetramethyl-1,3,2-dioxaboronic ester, catechol dioxaboronic ester, etc.); wherein R17 is an optionally substituted alkylene moiety of 1-6 carbon atoms.

The Scheme I reactants (a) and (b) are commercially available or can be prepared by means well known in the art. In general, the reactants (a) and at least one molar equivalent, and preferably a slight excess (e.g., 1.2 to 1.5 molar equivalents) of (b), as shown in Scheme I, are combined under standard reaction conditions in an inert solvent, such as dimethylformamide (DMF), at a temperature of about 25° C. until the reaction is complete, generally about 16 hours. Standard reaction conditions may comprise the use of a molar excess of suitable base, such as sodium or potassium hydroxide, triethylamine, diisopropylethylamine, N-methylmorpholine (NMM), or pyridine, or in some cases where LG is hydroxyl, a peptide coupling reagent, such as O-(7-azabenzotriazol-1-yl)-N,N,N′,N′-tetra methyluronium hexafluorophosphate (HATU), may be used. When the reaction is substantially complete, the product is subjected, if necessary, to a deprotection sequence under standard reaction conditions (e.g., THF, CH₂C₁₂, or the like, a molar excess of acid such as acetic acid, formic acid, trifluoroacetic acid, or the like as described herein) to yield isolated by conventional means. Further alternative synthetic methods for preparing compounds of Formula (I) are described in the synthetic sequences of Schemes II-V as disclosed in U.S. Pat. No. 8,558,001.

It will be appreciated that where typical or preferred process conditions (i.e., reaction temperatures, times, mole ratios of reactants, solvents, pressures, etc.) are disclosed in U.S. Pat. No. 8,558,001, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be necessary to prevent certain functional groups from undergoing undesired reactions. The term “protecting group” or “PG,” as used herein, is meant that a particular functional moiety, e.g., O, S, or N, is temporarily blocked so that a reaction can be carried out selectively at another reactive site in a multifunctional compound. “Protecting groups” or “PGs,” as used herein, are well known in the art and include those described in detail in Protective Groups in Organic Synthesis, Fourth Ed., Greene, T. W. and Wuts, P. G., Eds., John Wiley & Sons, New York: 2007, the entire contents of which are hereby incorporated by reference, and references cited therein.

The starting materials for the synthetic reaction Schemes I-V are as disclosed in U.S. Pat. No. 8,558,001 are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemie or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

Methods of Use in an Alcohol Consuming Patient Population

The present disclosure provides methods for treatment of addiction to a dopamine-producing agent comprising administering to a patient (e.g., a human in need of treatment) a therapeutically effective amount of an ALDH-2 inhibitor (e.g., compound of Formula (I)), wherein the patient is in a patient population that does not exclude alcohol consumption during treatment. These methods of treatment disclosed herein act to reduce addiction to the dopamine-producing agent in the patient, while allowing for alcohol consumption by the patient during treatment without the potentially serious cardiac side-effects that would require discontinuation of the treatment (e.g., the DER side-effects caused by alcohol consumption during treatment with DSF).

While not wishing to be bound by theory, ALDH-2 inhibitors of the present methods (such as the compounds of Formula (I)) are known to be effective in reducing or preventing surges in dopamine levels caused by administration of a substance containing a dopamine-producing agent. It is believed that, as a consequence of the ability of these ALDH-2 inhibitors to reduce surges in dopamine, they also reduce or prevent an addiction to a range of dopamine-producing agents such as alcohol, amphetamines, cocaine, nicotine, opioids, food, and other drugs of abuse.

It is contemplated that the methods of treatment disclosed herein can be used with any dopamine-producing agent associated with addiction for which a course of treatment is indicated. Thus, the methods of treatment of addiction to a dopamine-producing agent in a patient population that does not exclude alcohol during treatment as disclosed herein can be used as a method of treatment of addiction to amphetamines, alcohol, cocaine, nicotine, opioids, and other substances of abuse.

Generally, the present disclosure provides methods of treating addiction to a dopamine-producing agent, wherein the methods comprise administering to a patient (e.g., a human) in need thereof a therapeutically effective amount of an ALDH-2 inhibitor, and wherein the patient is a member of a patient population that does not exclude alcohol consumption during treatment. It is contemplated that in some embodiments of the method, the patient can consume alcohol in an amount consistent with “heavy drinking”—i.e., up to 5 drinks (e.g., 70 g EtOH) for males or 4 drinks (e.g., 56 g EtOH) for females —during treatment for addiction with an ALDH-2 inhibitor (e.g., up to 600 mg compound (2). It is also contemplated that the patient may consume this alcohol within about 1 hour, about 2 hours, about 3 hours, about 4 hours, or about 5 hours of administration of the ALDH-2 inhibitor and still suffer no serious side-effects associated with the alcohol consumption.

In various embodiments of the method, it is contemplated that a therapeutically effective amount of an ALDH-2 inhibitor (e.g., up to 600 mg compound (2)) can be administered to the patient and the patient can concomitantly consume alcohol in an amount of at least about 14 g, at least about 28 g, at least about 42 g, at least about 56 g, or at least about 70 g, without experiencing serious cardiac side-effects that would require discontinuation of the treatment. Indeed, it is contemplated in the methods of treatment of the present disclosure can be carried out wherein the patient consumes alcohol in an amount of about 14 g to about 42 g (i.e., about 1 to about 3 drinks), about 14 g to about 56 g (i.e., about 1 to about 4 drinks) or about 14 g to about 70 g (i.e., about 1 to about 5 drinks).

As noted above, it is a surprising advantage of the methods of the present disclosure, that relatively large therapeutically effective doses of the ALDH-2 inhibitor can be administered without the alcohol consuming patient experiencing the type of serious cardiac side-effects (e.g., high heart rate, low blood pressure) that were thought to result from acetaldehyde build-up due to inhibition of hepatic ALDH-2. As shown in the Examples of the present disclosure, up to 600 mg doses were administered to a population of healthy males who then consumed 5 drinks (i.e., 70 g EtOH) without occurrence of serious cardiac side-effects that would result in discontinuation of treatment. Thus, it is contemplated that the methods of treatment of the present disclosure can be carried out wherein the therapeutically effective amount of the ALDH-2 inhibitor administered to the patient can be in a broad range of dosages including from about 25 mg to about 600 mg, about 50 mg to about 600 mg, about 25 mg to about 400 mg, about 25 mg to about 200 mg. Moreover, it is contemplated that in some embodiments the amount of ALDH-2 inhibitor administered to the patient is at least 25 mg, at least 50 mg, at least 100 mg, at least 200 mg, at least 400 mg, or at least 600 mg.

It is contemplated that in some embodiments of the method, the administration of the therapeutically effective dose of the ALDH-2 inhibitor occurs prior to the patient consuming alcohol. It is also contemplated that in some cases the administration of the therapeutically effective dose of the ALDH-2 inhibitor occurs after the patient has consumed some amount of alcohol.

It is contemplated that in some embodiments of the methods administration of the ALDH-2 inhibitor comprises administering a therapeutically effective dose once-a-day. Thus, it is contemplated that the ALDH-2 inhibitor can be formulated as a once-a-day dose. In some embodiments, the once-a-day dose is in a formulation (e.g., a tablet), that is self-administered by the subject or patient.

In some embodiment of the methods, it is contemplated that the therapeutically effective dose of the ALDH-2 inhibitor can be in a unit dosage form. In some embodiments, the unit dosage of the ALDH-2 inhibitor (e.g., compound (2)) is an amount of about 25 mg to about 600 mg, about 50 mg to about 600 mg, about 25 mg to about 400 mg, or about 25 mg to about 200 mg. In some embodiments, the unit dosage ALDH-2 inhibitor (e.g., compound (2)) is an amount of about 25 mg, about 50 mg, about 100 mg, about 200 mg, about 300 mg, about 400 mg, about 500 mg, or about 600 mg. Further, it is contemplated that the patient could self-administer the unit dosage form of the ALDH-2 inhibitor.

In some embodiments, it is contemplated that the therapeutically effective dose of the ALDH-2 inhibitor (e.g., compound (2)) can be in an oral dosage form (e.g., a tablet). In some embodiments, the oral dosage form can be a unit dosage form comprising a therapeutically effective amount of an ALDH-2 inhibitor (e.g., compound (2)), wherein the amount of the ALDH-2 inhibitor is about 25 mg, about 50 mg, about 100 mg, about 200 mg, about 300 mg, about 400 mg, about 500 mg, or about 600 mg. Further, it is contemplated that the patient could self-administer the oral dosage form of the ALDH-2 inhibitor.

In some embodiments of the methods, it is contemplated that the ALDH-2 inhibitor is in the form of a pharmaceutical composition comprising the therapeutically effective dose of the ALDH-2 inhibitor compound, as well as a pharmaceutically acceptable carrier. Thus, in some embodiments of the method the administration can comprise self-administration of a pharmaceutical composition, wherein the pharmaceutical composition comprises a unit dosage form and/or an oral dosage form of an ALDH-2 inhibitor (e.g., a single tablet containing 25 mg or 100 mg of compound (2)).

As described above, in some embodiments of the methods, the patient can self-administer the pharmaceutically effective amount of the ALDH-2 inhibitor. Accordingly, in another aspect the present disclosure provides a patient pack comprising at least one pharmaceutical composition that comprises the ALDH-2 inhibitor and an information package or a product insert containing directions on the method of using the pharmaceutical composition.

Pharmaceutical Compositions

In some embodiments of the methods of the present disclosure, it is contemplated that the ALDH-2 inhibitor is administered in the form of a pharmaceutical composition. The pharmaceutical composition comprising an ALDH-2 inhibitor includes a dosage comprising a therapeutically effective amount of the active ingredient (e.g., compound (2)), or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants.

As disclosed elsewhere herein, in some embodiments of the methods the step of administering can comprise administering a pharmaceutical composition, wherein the pharmaceutical composition contains the ALDH-2 inhibitor (e.g., compound (2)) and a pharmaceutically acceptable carrier. Accordingly, in some embodiments the present disclosure also provides a pharmaceutical composition, wherein the composition comprises a therapeutically effective amount of an ALDH-2 inhibitor and a pharmaceutically acceptable carrier.

In some embodiments, the pharmaceutical composition useful in the methods of the present disclosure is in a unit dosage form, such as a dosage form that contains the active ingredient (e.g., compound (2)) in a single dosage form.

In some embodiments, the present disclosure provides a dosage form comprising a pharmaceutical composition of an ALDH-2 inhibitor (e.g., compound (2)) and a pharmaceutically acceptable carrier, wherein the dosage form comprises ALDH-2 inhibitor in a therapeutically effective amount.

In some embodiments, the pharmaceutical composition comprises a dosage of an ALDH-2 inhibitor of Formula (I) in an amount of about 25 mg to about 600 mg, about 50 mg to about 600 mg, about 25 mg to about 400 mg, or about 25 mg to about 200 mg. In some embodiments, the pharmaceutical composition comprises a dosage of an ALDH-2 inhibitor of Formula (I) in an amount of about 25 mg, about 50 mg, about 100 mg, about 200 mg, about 300 mg, about 400 mg, about 500 mg, or about 600 mg.

Such pharmaceutical compositions can be prepared using methods well known in the pharmaceutical art (see, e.g., Remington's Pharmaceutical Sciences, Mace Publishing Co., Philadelphia, Pa. 17th Ed. (1985) and Modern Pharmaceutics, Marcel Dekker, Inc. 3rd Ed. (G. S. Banker & C. T. Rhodes, Eds.). Methods of preparing pharmaceutical compositions of ALDH-2 inhibitor compounds, such as compounds of Formula (I), are described in e.g., U.S. Pat. Nos. 7,951,813, 8,158,810, 8,673,966, 8,558,001, 8,575,353, 9,000,015, and 9,610,299, each of which is hereby incorporated by reference herein.

Modes of Administering ALDH-2 Inhibitors

In the methods of the present disclosure it is contemplated that the pharmaceutical composition(s) the ALDH-2 inhibitor, such as a compound of Formula (I) (e.g., compound (2)), can be administered either as single or multiple doses, and by any of the accepted modes of administration of active ingredients having similar utility. For example, as described in U.S. Pat. No. 8,558,001, a pharmaceutical composition comprising an ALDH-2 inhibitor compound of Formula (I) can be administered using a variety of different modes including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One exemplary route for administering that is useful in the methods of the present disclosure is oral. Oral administration may be via capsule, enteric coated tablets, or the like. Typically, in making the pharmaceutical compositions that include a medication containing an ALDH-2 inhibitor, such as compound of Formula (I), the active ingredient(s) is diluted by an excipient and/or enclosed within a carrier in the form of a capsule, sachet, paper or other container. When the excipient serves as a diluent, it can be a solid, semi-solid, or liquid material (as above), which acts as a vehicle, carrier or medium for the active ingredient. Thus, the pharmaceutical composition(s) suitable for administering in the methods of the disclosure can be in the dosage form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Suitable excipients for use in the pharmaceutical compositions comprising ALDH-2 inhibitors of the present disclosure are well known in the art and include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Exemplary methods of preparing pharmaceutical compositions of ALDH-2 inhibitors suitable for use in the methods of the present disclosure are provided in the Examples.

The pharmaceutical compositions comprising ALDH-2 inhibitors useful in the methods of the present disclosure can be formulated so as to provide quick, sustained or delayed release of the relevant active ingredient after administration by employing procedures known in the art. Controlled release drug delivery systems for oral administration include osmotic pump systems and dissolutional systems containing polymer-coated reservoirs or drug-polymer matrix formulations. Examples of controlled release systems are given in e.g., U.S. Pat. Nos. 3,845,770; 4,326,525; 4,902,514; and 5,616,345.

The pharmaceutical compositions comprising ALDH-2 inhibitors useful in the methods of the present disclosure can also be formulated for administration via transdermal delivery devices (e.g., “patches”). Such transdermal patches may be used to provide continuous or discontinuous infusion of the pharmaceutical compositions in controlled amounts. The construction and use of transdermal patches for the delivery of pharmaceutical compositions is well known in the art. See, e.g., U.S. Pat. Nos. 5,023,252, 4,992,445 and 5,001,139. Such patches may be constructed for continuous, pulsatile, or on demand delivery of the pharmaceutical composition(s).

In some embodiments, the pharmaceutical composition(s) useful in the methods of the present disclosure are formulated in a unit dosage form.

The ALDH-2 inhibitor compounds useful in the methods of the present disclosure, e.g., compound of Formula (I), such as compound (2), are effective over a wide range of dosages and generally, are administered as a pharmaceutical composition in a pharmaceutically effective amount. In some embodiments, for oral administration, each dosage unit contains from about 10 mg to 1 g of an ALDH-2 inhibitor compound, such as compound of Formula (I), in some embodiments from 25 mg to 600 mg. In some embodiments, for parenteral administration, from 10 to 700 mg of an ALDH-2 inhibitor compound, such as compound of Formula (I), or in some embodiments, from about 50 mg to 300 mg.

Generally, in the methods of the disclosure, the amount of the ALDH-2 inhibitor compound, such as compound of Formula (I), to be administered will be determined by a physician, in view of relevant circumstances of the subject being so treated, the chosen route of administration, and of course, the age, the weight, the severity of symptoms, the response of the individual subject to the treatment, and the like.

For preparing a solid pharmaceutical composition useful in the methods of the present disclosure, the active ingredient is mixed with a pharmaceutical excipient to form a solid preformulation composition containing a homogeneous mixture of the active ingredient and the excipients. When referring to these preformulation compositions as homogeneous, it is meant that the active ingredient is dispersed evenly throughout the composition so that the composition may be readily subdivided into equally effective unit dosage forms such as tablets, pills and capsules. Tablets or pills may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Another exemplary mode for administering useful in the methods of the present disclosure is parenteral, particularly by injection. Pharmaceutical compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the active ingredients of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the known methods of preparation include vacuum-drying and freeze-drying techniques which yield a powder of the active ingredient plus any additional desired ingredient from a previously sterile-filtered solution thereof.

Pharmaceutical compositions that can be administered by inhalation or insufflation include solutions and suspensions in pharmaceutically acceptable, aqueous organic solvents, or mixtures thereof, and powders. The liquid or solid compositions may contain suitable pharmaceutically acceptable excipients as described herein and as known in the art. In some embodiments, the pharmaceutical composition of the ALDH-2 inhibitor (e.g., compound (2)) can be administered by the oral or nasal respiratory route for local or systemic effect. In some embodiments, the pharmaceutical compositions are prepared in pharmaceutically acceptable solvents which can be nebulized by use of inert gases. These nebulized solutions can be inhaled directly from the nebulizing device or the nebulizing device may be attached to a face mask tent, or intermittent positive pressure breathing machine. In some embodiments, the pharmaceutical compositions useful in the methods can be in solution, suspension, or powder compositions and can be administered, orally or nasally, from devices that deliver the formulation in an appropriate manner.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of Compound (1)-2,6-dichloro-N-[4-(2-oxo-1,2-dihydro-pyridin-4-yl)-benzyl]-benzamide

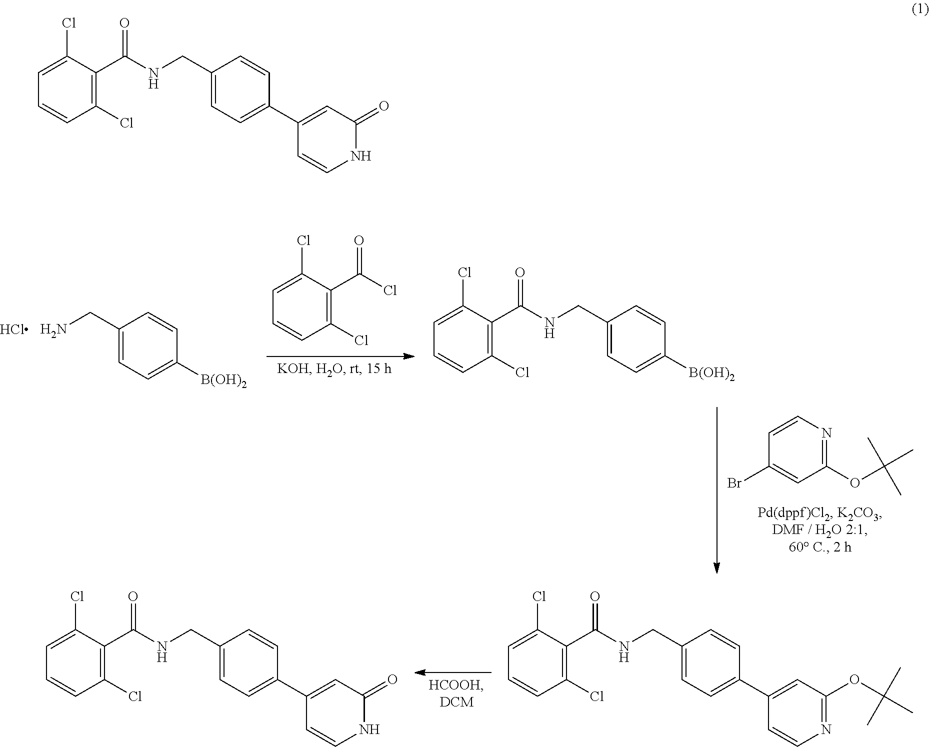

### Step 1—Preparation of 4-[(2,6-dichloro-benzoylamino)methyl]phenylboronic acid

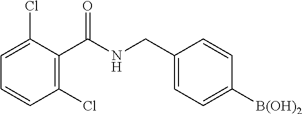 [SMILES: O=C(NCc1ccc(B(O)O)cc1)c1c(Cl)cccc1Cl]

4-(Aminomethyl)phenylboronic acid hydrochloride (5 g, 26.7 mmol) was dissolved in 25 mL water. 16 mL 50% aqueous KOH solution was added followed by 2,6-dichlorobenzoyl chloride (6.7 g, 32 mmol). The mixture was stirred rapidly at room temperature overnight. Acidification with 1N HCl gave a thick, white precipitate which was filtered, washed with water and dried giving 4-[(2,6-dichloro-benzoylamino) methyl]phenylboronic acid as a white powder in quantitative yield.

### Step 2—Preparation of N-[4-(2-tert-butoxy-pyridin-4-yl)-benzyl]-2,6-dichloro-benzamide

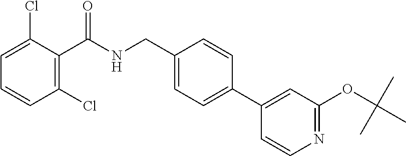 [SMILES: CC(C)(C)Oc1cc(-c2ccc(CNC(=O)c3c(Cl)cccc3Cl)cc2)ccn1]

4-[(2,6-Dichloro-benzoylamino)methyl]phenylboronic acid (5 g, 15.4 mmol), potassium carbonate (5 g), and [1,1′ bis(diphenylphosphino)ferrocene] dichloropalladium (II) (0.56 g, 0.77 mmol) were combined in a round bottom flask. 4-Bromo-2-(t-butoxy) pyridine (3.55 g, 15.4 mmol) was dissolved in 20 mL DMF and added to the flask under stirring. The flask was flushed with nitrogen and 10 mL water was added. The reaction mixture was stirred at 70° C. for two hours. After cooling the mixture was poured into 300 mL ethyl acetate and washed with water and brine. The organic phase was dried with magnesium sulfate and evaporated under vacuum. The crude N-[4-(2-tert-butoxy-pyridin-4-yl)-benzyl]-2,6-dichloro-benzamide was further purified by silica gel chromatography (eluent: hexane/ethyl acetate 1:1).

### Step 3—Preparation of 2,6-Dichloro-N-[4-(2-oxo-1,2-dihydro-pyridin-4-yl)-benzyl]-benzamide

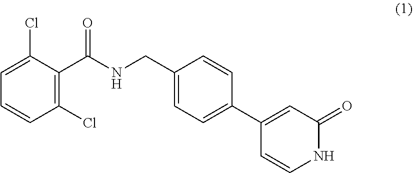 [SMILES: O=C(NCc1ccc(-c2cc[nH]c(=O)c2)cc1)c1c(Cl)cccc1Cl]

N-[4-(2-tert-Butoxy-pyridin-4-yl)-benzyl]-2,6-dichloro-benzamide was dissolved in 30 mL dichloromethane and 12 mL of 98% formic acid. The mixture was stirred at 40° C. for three hours after which the volatile components were evaporated under vacuum. The residue was triturated with ethyl acetate, filtered, washed with ethyl acetate and dried giving 2,6-dichloro-N-[4-(2-oxo-1,2-dihydro-pyridin-4-yl)-benzyl]-benzamide (4.34 g, 75.5% yield over two steps) as white powder. C₁₉H₁₄Cl₂N₂O₂: MS m/z: 373 (MH⁺) ¹H NMR (DMSO-d₆): δ 11.56 (s, 1H), δ 9.21 (t, J=5.6 Hz, 1H), δ 7.67 (d, J=8.0 Hz, 2H), δ 7.46 (m, 6H), δ 6.57 (d, J=1.2 Hz, 1H), δ 6.49 (dd, J=6.8 Hz, J′=1.6 Hz, 1H), δ 4.50 (d, J=6.0 Hz, 2H.

### Example 2: Preparation of Compound (2)-phosphoric acid mono-(4-{4-[(2,6-dichloro-benzoylamino)-methyl]-phenyl}-2-oxo-2H-pyridin-1-ylmethyl) ester

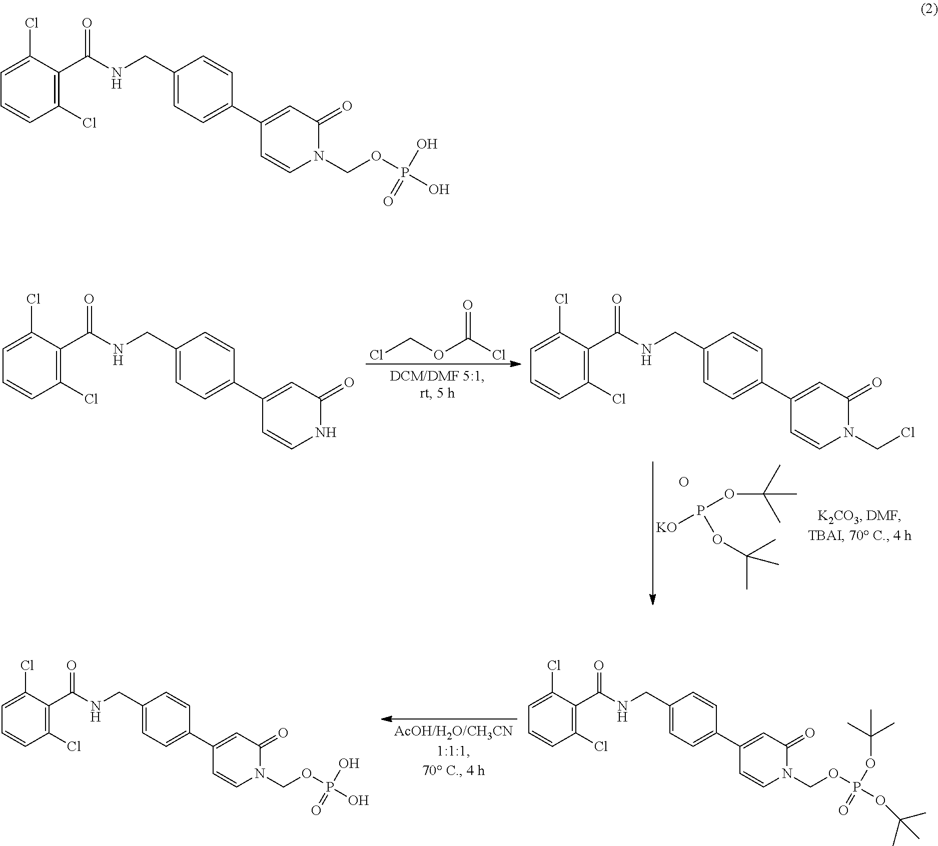

### Step 1—Preparation of 2,6-dichloro-N-[4-(1-chloromethyl-2-oxo-1,2-dihydro-pyridin-4-yl)-benzyl]-benzamide

2,6-Dichloro-N-[4-(2-oxo-1,2-dihydro-pyridin-4-yl)-benzyl]-benzamide (1.62 g, 4.34 mmol) (compound (1)), was suspended in 15 mL dichloromethane. Chloromethyl chloroformate (0.672 g, 5.21 mmol) was added followed by 3 mL DMF. The mixture was stirred at room temperature for five hours. After diluting with 200 mL ethyl acetate, the organic phase was washed with saturated, aqueous sodium bicarbonate solution and brine, dried with magnesium sulfate and evaporated under vacuum. The crude 2,6-dichloro-N-[4-(1-chloromethyl-2-oxo-1,2-dihydro-pyridin-4-yl)-benzyl]-benzamide was used in the following step without further purification.

### Step 2—Preparation of phosphoric acid di-tert-butyl ester 4-{4-[(2,6-dichloro-benzoylamino)-methyl]-phenyl}-2-oxo-2H-pyridin-1-ylmethyl ester

2,6-Dichloro-N-[4-(1-chloromethyl-2-oxo-1,2-dihydro-pyridin-4-yl)-benzyl]-benzamide from the previous step was dissolved in 50 mL DMF. Potassium carbonate (1 g) was added followed by potassium di(t-butyl)phosphate (2 g) and tetrabutylammonium iodide (50 mg). The mixture was stirred at 70° C. for four hours after which it was poured into 300 mL ethyl acetate. The organic phase was washed with water and brine, dried with magnesium sulfate and evaporated under vacuum. The crude product was further purified by silica gel chromatography (eluent: ethyl acetate), giving phosphoric acid di-tert-butyl ester 4-{4-[(2,6-dichloro-benzoylamino)-methyl]-phenyl}-2-oxo-2H-pyridin-1-ylmethyl ester as a colorless oil which slowly crystallized.

### Step 3—Preparation of phosphoric acid mono-(4-{4-[(2,6-dichloro-benzoylamino)-methyl]-phenyl}-2-oxo-2H-pyridin-1-ylmethyl) ester

Phosphoric acid di-tert-butyl ester 4-{4-[(2,6-dichloro-benzoylamino)-methyl]-phenyl}-2-oxo-2H-pyridin-1-ylmethyl ester from the previous step was dissolved in 20 mL acetonitrile, 20 mL acetic acid and 20 mL water, and heated at 70° C. for four hours. All volatile components were evaporated under vacuum and the residue was dissolved in 10 mL DMF. Slow addition of acetonitrile (˜60 mL) precipitated the product which was filtered, washed with more acetonitrile and dried, giving phosphoric acid mono-(4-{4-[(2,6-dichloro-benzoylamino)-methyl]-phenyl}-2-oxo-2H-pyridin-1-ylmethyl) ester (1.17 g, 56% over three steps) as a white powder. ¹H-NMR (DMSO) δ: 9.23 (t, J=6.2 Hz, 1H), 7.73 (d, J=8.4 Hz, 2H), 7.71 (d, J=8.4 Hz, 1H), 7.52-7.40 (m, 5H), 6.72 (d, J=1.6 Hz, 1H), 6.65 (dd, J=7.2 Hz, J=1.6 Hz, 1H), 5.61 (d, J=9.6 Hz, 2H), 4.52 (d, J=6.4 Hz, 2H). MS: 483/485 (MH⁺).

### Example 3: Formulation of Pharmaceutical Compositions

This example illustrates formulations of the pharmaceutical compositions comprising ALDH-2 inhibitors of formula (I) that can be used in the methods of the present disclosure for treating addiction to a dopamine-producing agent.

Hard gelatin capsules: The ingredients listed below are mixed and filled into hard gelatin capsules:

| | Quantity |
|---|---|
| Ingredient | (mg/capsule) |
| Active Ingredient | 30.0 |
| Starch | 305.0 |
| Magnesium stearate | 5.0 |

240 mg Tablets: The ingredients listed below are blended and compressed to form 240 mg tablets:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

120 mg Tablets: The ingredients listed below are blended and compressed as described below to form 120 mg tablets:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | |
| (as 10% solution in sterile water) | 4.0 mg |
| Sodium carboxymethyl starch | 4.5 mg |
| Magnesium stearate | 0.5 mg |
| Talc | 1.0 mg |
| Total | 120 mg |

The active ingredient, starch, and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Suppositories: Suppositories each containing 25 mg of active ingredient, are made as follows:

| Ingredient | Quantity |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions: A suspension containing 50 mg of active ingredient per 5.0 mL dose, is made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Subcutaneous: a subcutaneous formulation is prepared as follows:

| Ingredient | Quantity |
|---|---|
| Active Ingredient | 5.0 mg |
| Corn Oil | 1.0 mL |

Injectable: an injectable formulation is prepared by combining the following ingredients:

| Ingredient | Quantity |
|---|---|
| Active ingredient | 2.0 mg/mL |
| Mannitol, USP | 50 mg/mL |
| Gluconic acid, USP | q.s. (pH 5-6) |
| Water (distilled, sterile) | q.s. to 1.0 mL |
| Nitrogen Gas, NF | q.s. |

Topical: a topical preparation is prepared by combining the following ingredients as described below:

| Ingredients | Quantity (g) |
|---|---|
| Active ingredient | 0.01-1 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### Example 4: Dose-Ranging Study to Evaluate the Safety of Coadministration of ALDH-2 Inhibitor of Compound (2) and Ethanol in Healthy Male Alcohol Drinkers

This example illustrates a Phase 1 clinical study of the safety of coadministering the ALDH-2 inhibitor of compound (2) and ethanol in a population of healthy males.

### I. Objectives

The primary objective of the study was to evaluate the safety and tolerability of ascending doses of compound (2) when coadministered with ethanol (EtOH) in healthy male moderate drinkers. The exploratory objectives of the study were: (1) to evaluate the pharmacodynamic effects of single ascending doses of compound (2) when coadministered with EtOH in healthy male moderate drinkers; and (2) to evaluate the pharmacokinetics of single ascending doses of compound (2) when coadministered with EtOH in healthy male moderate drinkers, and to explore pharmacokinetic/pharmacodynamic relationships

### II. Methodology

This was a single-center, randomized, double-blind, placebo-controlled, single-ascending-dose cohort study to evaluate the safety and tolerability of the coadministration of up to 6 dose levels of compound (2) (25 mg, 50 mg, 100 mg, 200 mg, 400 mg and 600 mg) and EtOH in healthy male alcohol drinkers.

Subjects participated in a medical screening visit (Visit 1), a 1-day Qualification Phase (Visit 2), a 4-day Treatment Phase (Visit 3), and a follow-up visit (Visit 4).

Within 27 days of the medical screening visit, subjects were enrolled and attended an inpatient Qualification Phase in which they received up to 5 standard drinks (14 grams of EtOH each; oral solution of vodka containing 40% [v/v] EtOH, mixed with a non-carbonated beverage, one drink every 30 minutes) to evaluate their tolerance to repeat administration to ensure they could safely tolerate the planned EtOH treatment. A minimum washout interval of 44 hours was performed between initiation of the last EtOH administration in the Qualification Phase and study drug administration in the Treatment Phase.

Following qualification, eligible subjects were enrolled and randomized to receive compound (2) or placebo in a 3:1 fashion in each of 6 cohorts (8 subjects per cohort). As a safety precaution, no more than 4 subjects were administered study drug on any given day. Planned compound (2) dose levels included doses of 25 mg, 50 mg, 100 mg, 200 mg, 400 mg, and 600 mg. Study drug administration occurred approximately 1 hour following ingestion of a standardized (moderate-fat) meal. Approximately 2 hours after receiving the study drug, subjects began a session of repeat EtOH administration, during which they could receive up to 5 doses of EtOH (14 grams each, approximately 1 standard drink) every 30 minutes, or until a stopping criterion applied. Safety assessments, pharmacodynamic assessments and pharmacokinetic blood sample collections were obtained up to 48 hours post-dose. Subjects were discharged after 48-hour post-dose procedures were completed and the investigator deemed it safe to do so. Subjects returned for the safety follow-up visit approximately 7 (±2) days following discharge from the Treatment Phase or after early discontinuation from the study.

Dose escalation was determined based upon the safety results of preceding dose levels (up to a maximum of 600 mg). Following completion of each cohort, blinded safety data were reviewed by the investigator and medical monitor in order to determine if it was safe to escalate to the next dose level.

### III. Number of Subjects

A total of up to approximately 48 subjects were planned to be randomized to the Treatment Phase, with 8 subjects enrolled into and randomized within each of the 6 planned cohorts. A total of 48 subjects were randomized into the Treatment Phase (8 subjects per cohort) as planned, and all 48 subjects completed the planned treatments.

### IV. Main Criteria for Inclusion

Subjects were healthy male adults, between 21 and 45 years of age, inclusive, who were current alcohol users. Current alcohol user was defined as an individual who consumed alcohol during a typical week, and in the last 6 months consumed and tolerated ≥3 standard alcoholic drinks in one sitting. Subjects also had to be able to tolerate 5 standard alcoholic drinks in a 2-hour time period in the Qualification Phase to be eligible for the Treatment Phase. Subjects were excluded if they were deemed medically unsuitable for participation in this study, or unlikely to comply with the study protocol for any reason.

### V. Test Product (Compound (2)) Dose and Mode of Administration

Compound (2) 25 mg was administered orally as one 25 mg tablet (M10150).

Compound (2) 50 mg was administered orally as two 25 mg tablets.

Compound (2) 100 mg was administered orally as one 100 mg tablet (M10149).

Compound (2) 200 mg was administered orally as two 100 mg tablets.

Compound (2) 400 mg was administered orally as four 100 mg tablets.

Compound (2) 600 mg was administered orally as six 100 mg tablets

### VI. Duration of Treatment

Each subject participated in the study for up to approximately 6 weeks, from screening to follow-up.

### VII. Criteria for Evaluation

A. Safety

The primary endpoints included frequency and severity of adverse events (AEs), ethanol reaction (ER) scores and flushing, laboratory values, vital signs, and electrocardiograms (ECGs).

B. Pharmacodynamics

The exploratory pharmacodynamic endpoints included maximum effect (Eₘₐₓ), maximum change from baseline (CFBₘₐₓ), time to Eₘₐₓ (TEₘₐₓ), and time-averaged area under the effect curve (TA_AUE), as applicable, derived from Modified 5-item Drug Effects Questionnaire (mDEQ-5) scores.

C. Pharmacokinetics

The exploratory pharmacokinetic endpoints included: (1) plasma concentrations of compound (1) (the active metabolite of compound (2)) and Breath Alcohol Concentration (BrAC); and (2) pharmacokinetic parameters for compound (1) (e.g., maximum observed plasma concentration [Cₘₐₓ], time to Cₘₐₓ [Tₘₐₓ], area under the concentration vs. time curve from time zero to last quantifiable concentration [AUCₗₐₛₜ], AUC extrapolated to infinity (AUC_{0-∞}), AUC_{last/∞}, terminal half-life [tin], as applicable).

### VIII. Statistical Methods

A. Analysis Populations

The study analysis populations were defined as follows.

(1) Randomized population: all subjects who were assigned a randomization number in the Treatment Phase.

(2) Safety population: all randomized subjects who received any study drug in the Treatment Phase.

(3) Pharmacokinetic population: all randomized subjects who received at least one dose of study drug during the Treatment Phase, had evaluable pharmacokinetic data, and had no protocol deviations or other circumstances that would have excluded them from analysis.

(4) Pharmacodynamic population: all randomized subjects who received at least one dose of study drug during the Treatment Phase, had evaluable pharmacodynamic data, and had no protocol deviations or other circumstances that would have excluded them from analysis.

(5) Pharmacokinetic/pharmacodynamic population: all randomized subjects who received at least one dose of study drug during the Treatment Phase, had evaluable pharmacokinetic and pharmacodynamic data, and had no protocol deviations or other circumstances that would have excluded them from analysis.

B. Disposition, Demographics, and Baseline Characteristics

Disposition of each analysis population was summarized using descriptive statistics. Data from subjects who discontinued from the study were also summarized by last treatment received and primary reason for discontinuation. All deviations were listed.

Demographic and background characteristics, including alcohol use history, were summarized using descriptive statistics. Medical history and prior medications were listed by subject.

C. Safety

Safety data were analyzed using the Safety population.

Adverse events were summarized by incidence, severity, and relationship to study drug.

Subjects' ER scores and flushing were summarized descriptively by timepoint, category and treatment. In addition, flushing, ER heat sensation and ER palpitation were compared between each of the active doses and placebo using a mixed-effect model for repeated measures (MMRM) analysis with treatment, visit, and treatment by visit as fixed effects and baseline as a covariate. The actual value was used as the dependent variable and visit included pre-EtOH, 30 minutes, 60 minutes, 90 minutes, 120 minutes, 150 minutes and 3 hours relative to the first EtOH administration in Treatment Phase. The variance-covariance matrix was assumed to be unstructured. If the procedure did not converge, then a compound symmetric variance-covariance matrix was to be used instead. Results were presented as least square means (LSMEANS), treatment differences in LSMEANS, 95% confidence intervals (CI) and p-values.

Vital signs measurements (respiratory rate, systolic and diastolic blood pressure, and heart rate) were summarized by treatment and visit. Heart rate and diastolic blood pressure were also compared between each of the active doses and placebo using an MMRM analysis with treatment, visit, and treatment by visit as fixed effects and baseline as a covariate.

Twelve-lead ECG results were summarized by timepoint and treatment, and frequencies (numbers and percentages) were calculated for the overall evaluation. Concomitant medications, clinical laboratory evaluations (raw data and change from baseline, as applicable) and physical examination findings were listed.

D. Pharmacodynamics

Pharmacodynamics were analyzed using the Pharmacodynamic population.

mDEQ-5 scores were summarized by timepoint and treatment using descriptive statistics. Derived endpoints (Eₘₐₓ, CFBₘₐₓ, TEₘₐₓ, TA_AUE, as applicable) were summarized by treatment using descriptive statistics. For each mDEQ-5-derived endpoint, an analysis of variance (ANOVA) was conducted to compare each of active doses to placebo with dose group as a factor. Results were presented in terms of LSMEANS, treatment differences in LSMEANS, 95% CI and p-values.

Ethanol consumption and number of EtOH doses consumed (i.e., number of drinks) during the repeat EtOH administration sessions were summarized using descriptive statistics.

E. Pharmacokinetics

Pharmacokinetic data were analyzed using the Pharmacokinetic population.

Plasma concentrations of compound (1) and BrAC were summarized and listed by treatment, subject and timepoint. Pharmacokinetic parameters derived for compound (1) were also summarized and listed by treatment and subject. Pharmacokinetic parameters were estimated using a non-compartmental approach with a log-linear terminal phase assumption. The trapezoidal rule was used to estimate the area under the curve (linear trapezoidal linear interpolation) and the terminal phase will be estimated by maximizing the coefficient of determination estimated from the log-linear regression model. However, AUC_{0-∞}, AUC_{last/∞}, λz and t_{1/2} parameters were to be estimated for individual concentration-time profiles only when the terminal log-linear phase could be reliably characterized.

Compound (1) pharmacokinetic parameters (Cₘₐₓ, AUCₗₐₛₜ, and AUC_{0-∞}) were to be assessed for proportionality if at least 3 active doses were investigated. Proportionality analysis was done using a power model. The power model was defined as:

ln(PK parameter)=α+β⋅ ln(Dose)+ε

where α is the intercept, β is the slope and ε is the error term.

A linear model with ln-transformed dose as a continuous effect was fitted. A point estimate and a 90% CI was derived for the slope (β). The parameter could be considered to be dose proportional if the 90% confidence interval of the slope β was within [1+ln(0.8)/ln(r), 1+ln(1.25)/ln(r)], where r=the highest dose studied/the lowest dose (Smith et al., “Confidence interval criteria for assessment of dose proportionality.” Pharm Res. 17 (10):1278-83 (2000)).

F. Pharmacokinetic/Pharmacodynamic Relationship

Drug dose/concentration-response relationships were analyzed using the Pharmacokinetic/Pharmacodynamic population. The relationship of ER maximum scores and mDEQ-5 items (Eₘₐₓ and CFBₘₐₓ, TEₘₐₓ, and TA_AUE, if applicable) with compound (1) pharmacokinetic parameters (Cₘₐₓ and AUCₗₐₛₜ) were explored graphically.

### IX. Summary of Results

A. Safety Results

All treatments were generally well tolerated in this study. There were no deaths or serious adverse events (SAEs) and no subject was discontinued due to an AE. Two (5.6%) compound (2)-treated subjects did not consume all 5 EtOH administrations due to treatment-emergent AEs (TEAEs).

A total of 32 (88.9%) compound (2)-treated subjects and nine (75.0%) placebo-treated subjects reported a TEAE. The highest incidence of TEAEs was observed with compound (2) 100 mg, 200 mg and 400 mg, with all subjects (six subjects total [100%]) reporting at least one TEAE, followed by compound (2) 600 mg and 50 mg (five subjects each [83.3%]), and placebo (nine subjects [75.0%]); the lowest incidence was observed with compound (2) 25 mg (four subjects [66.7%]).

The majority of TEAEs were mild in severity and judged to be related to study drug (i.e., compound (2) or EtOH); one (0.6%) severe TEAE was experienced by a subject administered compound (2) 100 mg.

The most commonly reported TEAEs (>20% [>2 subjects]) were flushing, headache, feeling hot, and feeling drunk. The incidence of flushing and feeling hot was higher following administration of compound (2), while that of feeling drunk was higher following placebo; the incidence of headache was reported at a similar incidence with compound (2) and placebo.

In general, there was no clear dose response for the most common TEAEs; however, flushing, headache and feeling drunk appeared to increase with increasing dose of compound (2), though feeling drunk was reported for only a small number of subjects.

All mean laboratory values were within normal ranges at baseline and follow-up, with the exception of a not clinically significant increase in mean creatine kinase at follow-up in the compound (2) 100 mg dose group, primarily driven by one subject who had an abnormal, but not clinically significant creatine kinase value of 1350 U/L (repeat 3 days later: 583 U/L). There were no TEAEs related to laboratory values.

Mean vital signs values were within normal ranges at the timepoints tested. Administration of compound (2) doses >25 mg resulted in increases in mean heart rate on Day 1 beginning 15 minutes after first EtOH administration; greater increases in heart rate were observed for higher compound (2) doses (i.e., 200 mg, 400 mg and 600 mg). As shown in Table 2 (below), statistically significant increases in heart rate for compound (2)-treated subjects compared with placebo-treated subjects began at 45 minutes after first EtOH administration and continued until the last measured timepoint (135 minutes after first EtOH administration). The range of statistically significant differences from placebo was +15.3 to +35.9 beats per minute for compound (2) at doses of 200 mg through 600 mg, and +14.7 to +19.4 beats per minute for compound (2) at doses of 50 mg and 100 mg.

TABLE 2
Heart Rate (beats/min)

| | | Compound (2) dose | | | | | | | |
|---|---|---|---|---|---|---|---|---|---|
| | | 25 mg | 50 mg | 100 mg | 200 mg | 400 mg | 600 mg | Overall | Pbo |
| | | (N = 6) | (N = 6) | (N = 6) | (N = 6) | (N = 6) | (N = 6) | (N = 36) | (N = 12) |
| Visit | | n | | | | | | | |
| (min) | Statistics | 6 | 6 | 6 | 6 | 6 | 6 | 36 | 12 |
| Pre-EtOH | Mean | 59.5 | 56.7 | 57.0 | 60.2 | 61.2 | 61.2 | 59.3 | 64.7 |
| | (SD) | (5.13) | (10.25) | (2.28) | (3.43) | (9.83) | (5.74) | (6.57) | (7.49) |
| 45 | Mean | 65.3 | 72.7 | 70.5 | 84.5 | 81.2 | 85.2 | 76.6 | 66.8 |
| | (SD) | (4.72) | (16.23) | (4.04) | (10.67) | (10.87) | (10.80) | (12.27) | (5.94) |
| 75 | Mean | 63.5 | 80.5 | 81.8 | 97.0 | 82.7 | 91.8 | 82.9 | 66.7 |
| | (SD) | (8.85) | (23.36) | (13.24) | (18.73) | (14.15) | (9.35) | (17.86) | (6.24) |
| 105 | Mean | 66.5 | 81.8 | 83.8 | 101.3 | 86.2 | 91.8 | 85.3 | 65.4 |
| | (SD) | (10.75) | (21.05) | (15.08) | (14.92) | (14.16) | (9.77) | (17.35) | (7.65) |
| 135 | Mean | 64.7 | 76.8 | 81.3 | 96.2 | 83.7 | 92.3 | 82.5 | 67.6 |
| | (SD) | (6.59) | (16.80) | (17.78) | (22.49) | (15.56) | (8.16) | (17.81) | (7.61) |

Five TEAEs of tachycardia were reported: one subject who received compound (2) 50 mg had a TEAE of tachyarrhythmia, three subjects who received compound (2) 200 mg had a TEAE of sinus tachycardia, and one subject who received compound (2) 200 mg had a TEAE of tachycardia. All TEAEs began after consuming EtOH. The TEAEs were judged to be mild in severity and at least possibly related to study drug (i.e., compound (2) or EtOH). One subject in the compound (2) 200 mg group also experienced a TEAE of tachypnea after consuming EtOH that was judged to be mild and possibly related to study drug (i.e., compound (2) or EtOH).

As shown in Tables 3 and 4 (below), there were no significant changes in diastolic or systolic blood pressure associated with the compound (2)-treated subjects compared with placebo-treated subjects after EtOH administration.

TABLE 3
Diastolic Blood Pressure (mmHg)

| | | Compound (2) dose | | | | | | | |
|---|---|---|---|---|---|---|---|---|---|
| | | 25 mg | 50 mg | 100 mg | 200 mg | 400 mg | 600 mg | Overall | Pbo |
| | | (N = 6) | (N = 6) | (N = 6) | (N = 6) | (N = 6) | (N = 6) | (N = 36) | (N = 12) |
| Visit | | n | | | | | | | |
| (min) | Statistics | 6 | 6 | 6 | 6 | 6 | 6 | 36 | 12 |
| Pre- | Mean | 73.5 | 74.5 | 71.5 | 73.8 | 71.7 | 75.2 | 73.4 | 72.4 |
| EtOH | (SD) | (5.01) | (3.56) | (4.04) | (6.43) | (4.63) | (5.71) | (4.82) | (7.65) |
| 15 | Mean | 76.0 | 73.3 | 72.0 | 76.5 | 71.3 | 75.0 | 74.0 | 75.5 |
| | (SD) | (7.32) | (3.50) | (4.34) | (3.94) | (4.46) | (6.96) | (5.29) | (5.57) |
| 45 | Mean | 75.5 | 75.2 | 70.2 | 72.5 | 71.8 | 75.0 | 73.4 | 76.3 |
| | (SD) | (8.64) | (2.56) | (4.40) | (4.81) | (3.25) | (6.03) | (5.34) | (8.09) |
| 75 | Mean | 75.8 | 73.8 | 70.7 | 69.8 | 70.5 | 74.2 | 72.5 | 73.3 |
| | (SD) | (8.23) | (5.74) | (3.98) | (5.31) | (1.64) | (6.31) | (5.65) | (7.90) |
| 105 | Mean | 74.0 | 71.8 | 69.5 | 68.0 | 72.2 | 72.0 | 71.3 | 75.8 |
| | (SD) | (4.82) | (6.18) | (3.02) | (8.92) | (3.66) | (4.77) | (5.53) | (6.43) |
| 135 | Mean | 75.0 | 78.7 | 68.2 | 69.3 | 71.0 | 74.8 | 72.8 | 73.8 |
| | (SD) | (5.51) | (7.09) | (3.97) | (8.16) | (1.79) | (5.19) | (6.43) | (6.00) |

TABLE 4
Systolic Blood Pressure (mmHg)

| | | Compound (2) dose | | | | | | | |
|---|---|---|---|---|---|---|---|---|---|
| | | 25 mg | 50 mg | 100 mg | 200 mg | 400 mg | 600 mg | Overall | Pbo |
| | | (N = 6) | (N = 6) | (N = 6) | (N = 6) | (N = 6) | (N = 6) | (N = 36) | (N = 12) |
| Visit | | n | | | | | | | |
| (min) | Statistics | 6 | 6 | 6 | 6 | 6 | 6 | 36 | 12 |
| Pre- | Mean | 117.2 | 115.7 | 115.7 | 119.8 | 115.3 | 120.2 | 117.3 | 115.6 |
| EtOH | (SD) | (7.81) | (7.42) | (8.21) | (7.65) | (4.23) | (4.96) | (6.69) | (7.97) |
| 15 | Mean | 120.3 | 116.7 | 119.2 | 121.7 | 112.8 | 122.3 | 118.8 | 118.7 |
| | (SD) | (9.31) | (8.29) | (8.16) | (5.43) | (4.02) | (10.44) | (8.04) | (7.94) |
| 45 | Mean | 120.5 | 119.5 | 120.0 | 124.5 | 120.0 | 127.3 | 122.0 | 119.5 |
| | (SD) | (9.85) | (8.02) | (10.86) | (10.89) | (8.12) | (10.03) | (9.46) | (8.22) |
| 75 | Mean | 120.7 | 117.5 | 120.2 | 125.7 | 119.3 | 130.2 | 122.3 | 118.7 |
| | (SD) | (11.20) | (11.04) | (9.58) | (8.45) | (4.23) | (9.52) | (9.66) | (7.74) |
| 105 | Mean | 118.2 | 117.5 | 125.3 | 124.0 | 118.5 | 126.8 | 121.7 | 118.3 |
| | (SD) | (10.15) | (10.09) | (9.35) | (6.78) | (3.02) | (11.20) | (9.06) | (9.82) |
| 135 | Mean | 119.0 | 123.7 | 114.8 | 124.0 | 118.8 | 127.7 | 121.3 | 116.9 |
| | (SD) | (8.15) | (19.69) | (6.37) | (8.32) | (4.62) | (12.19) | (11.10) | (8.16) |

As shown in Table 5 (below), the mean ECG interval measures (i.e., “QTcF interval”) were similar between baseline and follow-up and there were no TEAEs related to ECG values.

TABLE 5
QTcF Interval, Aggregate (msec)

| | | Compound (2) dose | | | | | | | |
|---|---|---|---|---|---|---|---|---|---|
| | | 25 mg | 50 mg | 100 mg | 200 mg | 400 mg | 600 mg | Overall | Pbo |
| | | (N = 6) | (N = 6) | (N = 6) | (N = 6) | (N = 6) | (N = 6) | (N = 36) | (N = 12) |
| | | n | | | | | | | |
| Visit | Statistics | 6 | 6 | 6 | 6 | 6 | 6 | 36 | 12 |
| Prior to initiation | Mean | 403.7 | 400.7 | 401.3 | 400.2 | 396.7 | 393.8 | 399.4 | 397.6 |
| of repeat EtOH | (SD) | (24.68) | (23.87) | (10.60) | (21.41) | (21.67) | (11.84) | (18.65) | (21.05) |
| administration | | | | | | | | | |
| session | | | | | | | | | |
| 1 hour after | Mean | 406.7 | 402.5 | 405.3 | 403.2 | 394.8 | 394.3 | 401.1 | 397.8 |
| initiation of | (SD) | (25.45) | (21.84) | (14.12) | (14.52) | (16.61) | (22.46) | (18.84) | (19.89) |
| repeat EtOH | | | | | | | | | |
| administration | | | | | | | | | |
| session | | | | | | | | | |

A number of clinically significant findings related to general appearance on physical examinations were reported, the majority of which were related to flushing observed on Day 1.

On the ER subjective assessment, compound (2)-treated subjects were more likely to report feeling heat sensation and feeling palpitations compared with placebo-treated subjects. Only a small number of subjects reported feeling breathless or headache, but these were also more commonly reported among compound (2)-treated subjects. There were no differences between placebo- and compound (2)-treated subjects on the nausea or vomiting subscales. There was no notable compound (1) exposure-ER response relationship.

Flushing was not observed in any subjects who received compound (2) 25 mg. Flushing was observed for all other compound (2) doses, with the most reports of severe flushing (Grade 4) observed between 45 minutes and 135 minutes after the first EtOH administration, corresponding to between 2 and 5 drinks consumed. Flushing was observed in far fewer subjects following placebo than with compound (2).

B. Pharmacodynamic Results

Peak scores for placebo were higher than scores for all compound (2) dose levels on VAS measures of feeling any alcohol effects, feeling drunk, and liking the effects of alcohol Similar peak scores were observed for compound (2) and placebo on urge to drink alcohol and disliking alcohol effects.

Compound (2) did not show a consistent dose response on any of the pharmacodynamic measures. On some measures, certain compound (2) dose levels were associated with higher or lower scores compared with placebo; however, these results were not consistent and were more likely related to the variability associated with the relatively small sample size per cohort. There was no notable compound (1) exposure-pharmacodynamic response relationship.

Two subjects did not complete all 5 doses of EtOH. The majority of subjects were able to consume all 5 doses of EtOH and all were able to complete the pharmacodynamic questionnaires.

C. Pharmacokinetic Results

Following a moderate-fat meal, peak (Cₘₐₓ) and overall (AUC) exposure to compound (1) increased with increasing dose of compound (2). The increases across the 50 mg and 600 mg dose range were relatively linear; however, exposure was proportionately lower for the 25 mg dose.

Based on the power model for assessing dose proportionality, peak and overall exposure to compound (1) did not increase in a compound (2) dose-proportional manner across the 25 mg to 600 mg dose range. While statistically significant, the majority of 90% CIs for the estimated slopes (β) contained 1.00.

Tₘₐₓ of compound (1) ranged between approximately 3.5 to 5 hours.

t_{1/2} of compound (1) was moderate (approximately 17 to 27 hours), with slightly longer t_{1/2} at the lowest compound (2) dose levels (25 mg and 50 mg) compared with higher dose levels.

Breath alcohol concentrations increased with each repeated EtOH administration, with peak EtOH concentrations generally occurring following the last (fifth) EtOH administration. Peak breath alcohol concentrations were slightly higher in compound (2)-treated groups compared with placebo.

### X. Conclusions

The results of this safety study suggest that administration of escalating doses of compound (2) in combination with multiple administrations of EtOH was relatively well tolerated in male alcohol drinkers. Although there was an increased risk of EtOH-related physiologic effects at higher doses of compound (2), the majority of subjects completed the repeat EtOH administration and no compound (2) dose escalation stopping criteria were met, suggesting that compound (2) at doses up to 600 mg and EtOH can be safely coadministered in this population.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the inventions.

What is claimed is:

1. A method of treating addiction to a dopamine-producing agent, the method comprising administering to a patient in need thereof, wherein the patient is a member of a patient population that does not exclude alcohol consumption during treatment, a therapeutically effective amount of an ALDH-2 inhibitor.

2. The method of claim 1, wherein the dopamine-producing agent is an agent other than alcohol; optionally, wherein the dopamine-producing agent is selected from amphetamine, cocaine, food, nicotine, opioids, or other drugs of addiction.

3. The method of claim 1, wherein the patient population does not exclude: (i) male patients that consume from 1 to 5 alcoholic drinks during treatment, or (ii) female patients that consume from 1 to 4 alcoholic drinks during treatment.

4. The method of claim 1, wherein the patient consumes alcohol during treatment; optionally, wherein the patient consumes alcohol within about 5 hours after administration of the ALDH-2 inhibitor.

5. The method of claim 4, wherein the patient consumes alcohol in an amount of about 14 g to about 70 g.

6. The method of claim 1, wherein the therapeutically effective amount of the ALDH-2 inhibitor is about 25 mg to about 600 mg.

7. The method of claim 1, wherein the ALDH-2 inhibitor is in a dosage form comprising the ALDH-2 inhibitor and a pharmaceutically acceptable carrier.

8. The method of claim 1, wherein the ALDH-2 inhibitor is in an oral dosage form.

9. The method of claim 1, wherein the ALDH-2 inhibitor is self-administered.

10. The method of claim 1, wherein the ALDH-2 inhibitor is a compound of Formula (I): 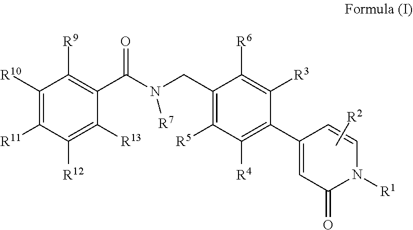
wherein:
R¹ is hydrogen, optionally substituted C₁₋₆ alkyl, —CH₂OH, —CH₂OP(O)(OR²⁰)(OR²¹);
R² is hydrogen, optionally substituted C₁₋₆ alkyl, cycloalkyl, or halo;
each of R³, R⁴, R⁵, R⁶, R⁹, R¹⁰, R¹¹, R¹² and R¹³ is independently hydrogen, hydroxyl, —OP(O)(OR²⁰)(OR²¹), —CH₂OH, —CH₂OP(O)(OR²⁰)(OR²¹), optionally substituted alkyl, optionally substituted alkylene, optionally substituted alkynyl, optionally substituted alkoxy, optionally substituted cycloalkyl, optionally substituted aryl, optionally substituted aralkyl, optionally substituted heteroaryl, optionally substituted heteroaralkyl, optionally substituted heterocyclyl, aminocarbonyl, acyl, acylamino, —O—(C₁ to C₆-alkyl)-O—(C₁ to C₆-alkyl), cyano, halo, —SO₂NR²⁴R²⁵; or —NR²⁴R²⁵;
R⁷ is hydrogen or optionally substituted C₁₋₆ alkyl;
each of R²⁰ and R²¹ is independently Na⁺, Li⁺, K⁺, hydrogen, C₁₋₆ alkyl; or R²⁰ and R²⁴ can be combined to represent a single divalent cation Zn²⁺, Ca²⁺, or Mg²⁺; and
each of R²⁴ and R²⁵ is independently chosen from hydrogen or C₁₋₆ alkyl or when combined together with the nitrogen to which they are attached form a heterocycle; or
a pharmaceutically acceptable salt, ester, single stereoisomer, mixture of stereoisomers, or a tautomer thereof.

11. The method of claim 10, wherein
R¹ is hydrogen, methyl, or —CH₂OP(O)(OR²⁰)(OR²¹);
R² is hydrogen, methyl, or fluoro;
each of R³ or R⁴ is independently hydrogen or methyl;
each of R⁵ and R⁶ is independently hydrogen or fluoro;
R⁷ is hydrogen;
R⁹ is hydrogen, chloro, fluoro, or methyl;
R¹⁰ is hydrogen or fluoro;
R¹¹ is hydrogen or —OCH₂CH₂OCH₃;
R¹² is hydrogen or fluoro;
R¹³ is hydrogen, chloro, fluoro, or methyl; and
each of R²⁰ and R²¹ is independently Na⁺, Li⁺, K⁺, or hydrogen.

12. The method of claim 11, wherein the compound of Formula (I) is selected from:
2,6-dichloro-N-[4-(2-oxo-1,2-dihydro-pyridin-4-yl)-benzyl]-benzamide;
phosphoric acid mono-(4-{4-[(2,6-dichloro-benzoylamino)-methyl]-phenyl}-2-oxo-2H-pyridin-1-ylmethyl) ester;
2,6-dichloro-4-(2-methoxyethoxy)-N-(4-(2-oxo-1,2-dihydropyridin-4-yl) benzyl)benzamide;
2-chloro-3-fluoro-N-(4-(2-oxo-1,2-dihydropyridin-4-yl)benzyl)benzamide;
2-chloro-6-methyl-N-(4-(2-oxo-1,2-dihydropyridin-4-yl)benzyl)benzamide;
2,6-dimethyl-N-(4-(2-oxo-1,2-dihydropyridin-4-yl)benzyl)benzamide;
2,6-dichloro-N-[4-(6-methyl-2-oxo-1,2-dihydro-pyridin-4-yl)-benzyl]-benzamide;
2-chloro-3,6-difluoro-N-(4-(2-oxo-1,2-dihydropyridin-4-yl)benzyl)benzamide;
2,6-dichloro-N-(3-methyl-4-(2-oxo-1,2-dihydropyridin-4-yl)benzyl)benzamide);
2,6-dichloro-N-(4-(1-methyl-2-oxo-1,2-dihydropyridin-4-yl)benzyl)benzamide;
2,6-difluoro-N-(4-(2-oxo-1,2-dihydropyridin-4-yl)benzyl)benzamide;
2-chloro-6-fluoro-N-(4-(2-oxo-1,2-dihydropyridin-4-yl)benzyl)benzamide;
2,6-dichloro-N-(2-fluoro-4-(2-oxo-1,2-dihydropyridin-4-yl)benzyl)benzamide;
2,6-dichloro-N-(4-(5-fluoro-2-oxo-1,2-dihydropyridin-4-yl)benzyl)benzamide;
2,6-dimethyl-N-(4-(2-oxopiperidin-4-yl)benzyl)benzamide;
or a pharmaceutically acceptable salt, single stereoisomer, mixture of stereoisomers, or a tautomer thereof.

13. The method of claim 11, wherein the compound of Formula (I) is compound (1): 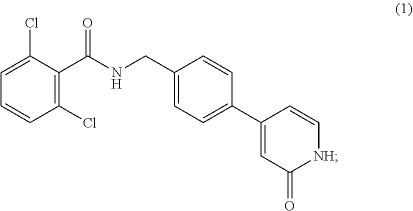 [SMILES: O=C(NCc1ccc(-c2cc[nH]c(=O)c2)cc1)c1c(Cl)cccc1Cl]
or a pharmaceutically acceptable salt, or a tautomer thereof.

14. The method of claim 11, wherein the compound of Formula (I) is compound (2): 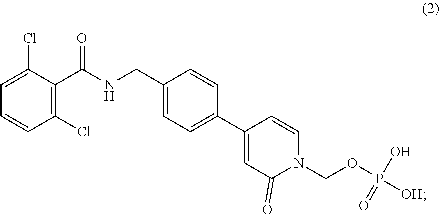 [SMILES: O=C(NCc1ccc(-c2ccn(COP(=O)(O)O)c(=O)c2)cc1)c1c(Cl)cccc1Cl]
or a pharmaceutically acceptable salt, ester, or a tautomer thereof.

15. The method of claim 1, wherein the ALDH-2 inhibitor is a compound comprising an isoflavone structure.

16. The method of claim 15, wherein the ALDH-2 inhibitor compound is daidzein (compound (15)): 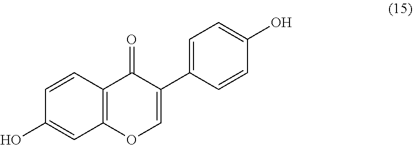 [SMILES: O=c1c(-c2ccc(O)cc2)coc2cc(O)ccc12]
or a pharmaceutically acceptable salt, ester, or a tautomer thereof.

17. The method of claim 15, wherein the ALDH-2 inhibitor compound is {[3-(4-aminophenyl)-4-oxochromen-7-yloxy]methyl}benzoic acid (compound (16)): 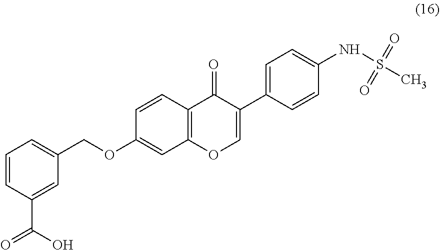 [SMILES: CS(=O)(=O)Nc1ccc(-c2coc3cc(OCc4cccc(C(=O)O)c4)ccc3c2=O)cc1]
or a pharmaceutically acceptable salt, ester, or a tautomer thereof.
